(19) United States
(12) Patent Application Publication
(10) Pub. No.: US 2008/0293745 A1
(43) Pub. Date: Nov. 27, 2008

(54) New amino-alkyl-amide derivatives as CCR3 receptor ligands

(75) Inventors: Agnes PAPPNE BEHR, Budapest (HU); Zoltan KAPUI, Budapest (HU); Peter ARANYI, Budapest (HU); Sandor BATORI, Budapest (HU); Veronika BARTANE BODOR, Budapest (HU); Lajos T. NAGY, (US); Mihalyne Santa, Mezotur (HU); Marton VARGA, Dunakeszi (HU); Gyorgy FERENCZY, Budapest (HU); Endre MIKUS, Budapest (HU); Katalin URBAN-SZABO, Budapest (HU); Judit VARGANE SZEREDI, Budapest (HU); Tibor SZABO, Budapest (HU); Erzsebet WALCZ, Budapest (HU)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/050,698

(22) Filed: Mar. 18, 2008

Related U.S. Application Data
(63) Continuation of application No. PCT/HU2006/000077, filed on Sep. 19, 2006.

(30) Foreign Application Priority Data
Sep. 22, 2005 (HU) P0500877

(51) Int. Cl. A61K 31/519; A61K 31/437; C07D 471/04; C07D 513/04; A61P 31/18; A61K 31/423; C07D 263/58

(52) U.S. Cl. 514/260.1; 546/114; 514/301; 514/375; 548/221; 544/255

(57) ABSTRACT

The invention relates to a compound of the general formula (I),

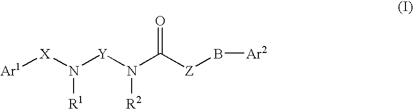

as defined herein which is useful for the treatment of a pathology in a patient wherein a CCR3 receptor plays a role in the development of the pathology, and pharmaceutical preparations containing such compound.

The invention is also directed to a process for preparing the compound of the general formula (I), and intermediate useful in the preparation.

### FIELD OF THE INVENTION

The present invention relates to the CCR3 receptor ligands of general formula (I), within them favourably antagonists and to the salts, solvates and isomers thereof, to the pharmaceutical compositions containing them, to the use of the compounds of the general formula (I) and their salts, solvates and isomers, to the preparation of the compounds of the general formula (I) and their salts, solvates and isomers and to the new intermediates of the general formula (III).

### BACKGROUND OF THE INVENTION

Chemokines are small molecular weight (8-12 kDa) secreted polypeptides playing important regulatory role in the immune processes due to their leukocyte attracting (chemotactic) effect. They exert their effects through the chemokine receptors, which belong to the family of the G protein coupled receptors.

The CC chemokine receptors 3 (CCR3 receptors) are expressed by a number of inflammatory cells, like the basofils, the mast cells, T lymphocytes, epithelial cells, dendritic cells, but they can be found in the greatest amount on the surface of the eozinofiles.

The CCR3 receptor ligands belong to the family of the C—C chemokines. They have a number of selective and non-selective ligands. The selective ligands are the eotaxin, eotaxin-2 and the lately discovered eotaxin-3. The non-selective ligands are the RANTES, the monocyte chemotactic proteins (MCP-2, MCP-3, MCP-4) and the macrophag inhibitor protein (MIP-1). The best characterized CCR3 ligand known from a long time is the eotaxin.

The eotaxin through the activation of the CCR3 receptors attracts selectively the eosinofils. Prior to an allergen provocation, the measured eotaxin level in the broncho-alveolar lavage fluidum of asthmatic patients was by 67 percent higher. On the effect of provocation a 2.4-fold increase of the epithelial and endothelial cells of the respiratory tract were found.

In the lung the eotaxin is produced in many cells. Following an allergen response, the most important eotaxin sources are the epithelial cells, but a great amount of eotaxin is produced by the fibroblasts of the lung, the smooth muscle cells and the endothelial cells of the respiratory tract, the alveolar macrophags and lymphocytes, and the eosinofils themselves.

Originally the data showed that the CCR3 receptors are to be found only in the eosinofil cells (Bertrand C P, Ponath P D., Expert Opin Investig Drugs. 2000 January; 9(1):43-52.), but on the basis of expression profiles it has been revealed that other inflammation cells—although in smaller amount—also contain CCR3 receptors (Elsner J, Escher S E, Forssmann U., Allergy. 2004 December; 59(12):1243-58.). Thus, the CCR3 antagonists possess much wider effect, their activity is not limited to the eosinofils and consequently they can be considered much more valuable and effective targets in the treatment of asthmatic, allergic and inflammatory diseases.

Based on the above observations, CCR3 antagonists may possess important profilactic and therapeutic effects in the treatment of pathologies where in the development of the disease CCR3 receptors play a role. These diseases are characterized by the disorder of the leucocyte functions (activation, chemotaxis), there are numerous chronic inflammatory diseases among them, such as asthma, allergic rhynitis, atopic dermatitis, eczema, inflammatory bowel disease, ulcerative colitis, allergic conjunctivitis, arthritis, multiple sclerosis, Crohn disease, HIV-infection and diseases in conjunction with AIDS.

The CCR3 antagonists published to date in the literature are carbamide-, thiocarbamide derivatives (WO 01/09088, WO 02/059081) and/or compounds containing saturated cyclic amino group (WO 00/35451, U.S. Pat. No. 6,605,623, WO 01/98270, WO 03/004487, WO 03/018556, WO 2004/028530, WO 00/53600, WO 00/35876, WO 01/64216, WO 02/50064, WO 02/102775, GB 2373186, WO 03/082291, WO 2004/004731, WO 2004/058702, WO 2004/085423 WO 2004/076448, WO 2004/084898). The present invention relates to a new structural type of compounds, to the open-chain amino-alkyl-amide derivatives, representatives of these compounds are effective CCR3 receptor antagonists.

From the aspect of therapeutic use it is essential that the molecules do not bind, or bind only in case of very high concentration to other the CCR receptor subtypes.

Our aim was to prepare compounds of high antagonistic activity, and at the same time selective to the CCR3 receptor, i.e. which inhibit the CCR3 receptor in much smaller concentration as compared to other CCR receptors. Further aim was that the new compounds have stability, bioavailability, therapeutic index and toxicity values, which ensure its drugability. Additional aim was that the compounds, through their good enteric absorption can be applied orally.

### SUMMARY OF THE INVENTION

We have found that the compounds of the general formula (I),

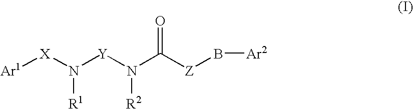

wherein
B stands for sulfur atom, or —SO— or —SO₂— group;
Ar¹ represents phenyl- or naphthyl group, optionally substituted with one or more identical or non-identical substituents selected from the group consisting of straight or branched C₁₋₄ alkyl group, halogen atom, straight or branched C₁₋₄ alkoxy group, trifluoromethyl group, cyano group, nitro group, hydroxyl group, C₁₋₂ alkylenedioxy group, amino group, and amino group—substituted with one or two identical or non-identical straight or branched C₁₋₄ alkyl group—;
X and Y independently mean a straight C₁₋₄ alkylene group optionally substituted with one or more identical or non-identical straight or branched C₁₋₄ alkyl group;
Z stands for a straight C₁₋₄ alkylene group optionally substituted with one or more identical or non-identical straight or branched C₁₋₄ alkyl group or phenyl group;
R¹ and R² independently mean hydrogen atom or a straight or branched C₁₋₄ alkyl group;
Ar² stands for phenyl-, benzyl-, thienyl- or furyl group, optionally substituted with one or more identical or non-identical substituents selected from the group consisting of straight or branched C₁₋₄ alkyl group, straight or branched C₁₋₄ alkoxy group, hydroxyl group, amino group, amino group—substituted with one or two identical or non-identical straight or branched C₁₋₄ alkyl or aralkyl group—, trifluoromethyl group, cyano group, C₁₋₂ alkylenedioxy group, and halogen atom;
   5- or 6-membered heterocyclic ring containing one, two, three or four nitrogen atoms, or two nitrogen atoms and one oxygen atom, or one nitrogen atom and one oxygen atom, or one nitrogen atom and one sulphur atom, optionally substituted with one or more identical or non-identical substituents selected from the group consisting of straight or branched C₁₋₄ alkyl group, C₃₋₆ cycloalkyl group, 1,4-butylene group, straight or branched C₁₋₄ alkoxy group, halogen atom, nitro group, cyano group, hydroxyl group, amino group, amino group—substituted with one or two identical or non-identical straight or branched C₁₋₄ alkyl—, aralkyl group, trifluoromethyl group, C₁₋₄ hydroxyalkyl group, phenyl group—optionally substituted with one or more straight or branched C₁₋₄ alkyl group, halogen atom or benzyloxy group—, benzyl group—optionally substituted with one or more straight or branched C₁₋₄ alkyl group, straight or branched C₁₋₄ alkoxy group or halogen atom—, furyl group, thienyl group, pyridyl group, —CO—O—R³-alkoxycarbonyl group—where R³ stands for straight or branched C₁₋₄ alkyl group—, —NH—CH₂—CO—O—R⁴ group—where R⁴ stands for straight or branched C₁₋₄ alkyl group—, —C₆H₄—NH—CO—R⁵ group—where R⁵ stands for straight or branched C₁₋₄ alkyl group—, and oxo group;
   benzologue of the 5- or 6-membered heterocyclic ring group wherein the benzene ring may optionally be further substituted with one or more identical or non-identical substituents selected from the group consisting of straight or branched C₁₋₄ alkyl group, straight or branched C₁₋₄ alkoxy group, hydroxyl group, trifluoromethyl group, cyano group, nitro group, C₁₋₂ alkylenedioxy group, amino group, amino group—substituted with one or two identical or non-identical straight or branched C₁₋₄ alkyl or aralkyl group—, halogen atom, sulfonyl group, and sulfonamide group;
   5- or 6-membered heterocyclic ring containing one, two or three nitrogen atoms, or one nitrogen atom and one oxygen atom, or one nitrogen atom and one sulphur atom, condensed with a 6-membered heteroaromatic ring group containing one or two nitrogen atoms, optionally substituted with one or more identical or non-identical substituents selected from the group consisting of straight or branched C₁₋₄ alkyl group, straight or branched C₁₋₄ alkoxy group, halogen atom, nitro group, cyano group, hydroxyl group, amino group, amino group—substituted with one or two, identical or non-identical straight or branched C₁₋₄ alkyl group or benzyl group—, and 1-(C₁₋₄-alkylcarbonyl)-2-phenylethyl group;
      with the proviso that if B stands for —SO₂— group and the meanings of Ar¹, X, Y, R¹, R² and Ar² are as defined above, Z means a straight C₁₋₄ alkylene group—optionally substituted with one or more identical or non-identical straight or branched C₁₋₄ alkyl group, and with the further proviso that when Ar¹ represents phenyl group, X means methylene group substituted with one methyl group, Y means propylene group, Z stands for propylene or butylene group, R¹ means methyl group, R² means hydrogen atom and Ar² stands for phenyl group, B is different from —SO₂— group;
and their salts, solvates and isomers and the salts and solvates thereof, fulfill the above criteria.

### DETAILED DESCRIPTION OF THE INVENTION

The detailed meanings of the above substituents are as follows:

by a C₁₋₄ alkyl group we mean a saturated straight- or branched-chain aliphatic group of 1-4 carbon atom, such as methyl-, ethyl-, propyl-, isopropyl-, butyl-, isobutyl-, secondary butyl-, tertiary butyl group.

by a C₁₋₄ alkylene group we mean a —(CH₂)ₙ— group where the value of n is 1, 2, 3 or 4, such as a methylene-, ethylene-, propylene-, butylene group.

by a C₃₋₆ cycloalkyl group we mean a cyclic alkyl group of 3-6 carbon atoms such as cyclopropyl-, cyclobutyl-, cyclopentyl-, cyclohexyl group.

by a C₁₋₄ alkoxy group we mean an —O-alkyl group—where the meaning of alkyl is as defined above—, such as methoxy-, ethoxy-, propoxy-, isopropoxy-, butoxy-, isobutoxy-, secondary butoxy-, tertiary butoxy group.

by a C₁₋₂ alkylenedioxy group we mean an —O-alkylene-O— group—where the meaning of alkylene is as defined above—, such methylenedioxy-, ethylenedioxy group.

by a C₁₋₄ hydroxyalkyl group we mean an alkyl group substituted with a hydroxyl group, —where the meaning of alkyl is as defined above, such as hydroxymethylene-, hydroxyethylene group.

by aralkyl group we mean a (C₁₋₄ alkyl)-phenyl group, —where the meaning of alkyl is as defined above—, and the phenyl group may be substituted with halogen atom, C₁₋₄ alkyl group, C₁₋₄ alkoxy group.

by halogen atom we mean chloro, fluoro, iodo or bromo atom.

by a 5- or 6-membered heterocyclic ring containing one, two or three nitrogen atoms we mean an unsaturated, saturated or partially saturated heterocyclic ring, for example pyrrole, imidazole, pyrazole, 1,2,3-triazole, 1,2,4-triazole, pyridine, pyrimidine, pyridazine, pyrazine 1,2,4-triazine, 1,3,5-triazine, 1,2,3-triazine, pyrrolidine, imidazolidine, [1,2,4]triazolidine, piperidine, piperazine, 2-imidazoline ring.

by a 5- or 6-membered heterocyclic ring containing one nitrogen atom and one oxygen or sulphur atom we mean an unsaturated, saturated or partially saturated heterocyclic ring, for example oxazole, isoxazole, thiazole, isothiazole, 1,2-oxazine, 1,3-oxazine, 1,4-oxazine, 1,2-thiazine, 1,3-thiazine, 1,4-thiazine, oxazolidine, thiazolidine, morpholine, thiomorpholine, 2-thiazoline, 2-oxazoline ring.

The heterocyclic ring containing two nitrogen atoms and one oxygen atom may be for example an oxadiazole ring.

By benzologue we mean derivatives condensed with benzene ring, for example indole, benzoxazole, benzthiazole, benzimidazole, quinoline, quinazoline, quinoxaline.

A derivative of a 5- or 6-membered heterocyclic ring—containing one, two or three nitrogen atoms, or one nitrogen atom and one oxygen atom, or one nitrogen atom and one sulphur atom—condensed with 6-membered heterocyclic rings—containing one or two nitrogen atom, may for example be a thiazolopyridine, triazolopyridine, thiazolopyrimidine, oxazolopyridine, 9H-purine, 3H-imidazopyridine.

By salts of the compounds of general formula (I) we mean salts given with inorganic and organic acids and bases. Favourable are the salts formed with pharmaceutically acceptable acids e.g. hydrochloric acid, sulfuric acid, ethanesulfonic acid, tartaric acid, fumaric acid, citric acid, and bases e.g. sodium hydroxide, potassium hydroxide, ethanolamine. The salts formed during the purification and isolation process, favourably with tetrafluoroboric acid and perchloric acid, are also subjects of the invention.

By solvates we mean solvates formed with various solvents, e.g. with water or ethanol.

By isomers we mean structural and optical isomers. Structural isomers may be tautomeric forms in equilibrium or isolated desmotrops, which are also subjects of the invention. The compounds of general formula (I) may contain one or more asymmetric carbon atom, thus they may be optical isomers, enantiomers or diastereoisomers. These enantiomers and diastereoisomers and the mixtures thereof, including the racemates are also subjects of the invention.

A favourable group of the compounds of general formula (I) is formed by the compounds, where
B stands for sulfur atom, —SO— or —SO₂— group;
Ar¹ stands for phenyl group, optionally substituted with one or more halogen atom;
X and Y independently mean a straight C₁₋₄ alkylene group, optionally substituted with one or more identical or non-identical straight or branched C₁₋₄ alkyl group;
Z stands for a straight chain C₁₋₄ alkylene group, optionally substituted with one or more identical or non-identical straight or branched C₁₋₄ alkyl group or phenyl group;
R¹ and R² independently mean hydrogen atom or a straight or branched C₁₋₄ alkyl group;
Ar² stands for phenyl group; or
   5- or 6-membered heterocyclic ring group containing one, two, three or four nitrogen atoms, or two nitrogen atoms and one oxygen atom, or one nitrogen atom and one oxygen atom, or one nitrogen atom and one sulphur atom, optionally substituted with one or more, identical or non-identical substituents selected from the group consisting of straight or branched C₁₋₄ alkyl group, C₃₋₆ cycloalkyl group, 1,4-butylene group, cyano group, amino group, trifluormethyl group, C₁₋₄ hydroxyalkyl group, phenyl group—optionally substituted with one or more straight or branched C₁₋₄ alkyl group, halogen atom or benzyloxy group—, benzyl group—optionally substituted with straight or branched C₁₋₄ alkoxy group or halogen atom—, thienyl group, furyl group, pyridyl group, —CO—O—R³-alkoxycarbonyl group—where R³ stands for straight or branched C₁₋₄ alkyl group—, —NH—CH₂—CO—O—R⁴ group—where R⁴ stands for straight or branched C₁₋₄ alkyl group—, —C₆H₄—NH—CO—R⁵ group—where R⁵ stands for straight or branched C₁₋₄ alkyl group—, oxo group;
   benzologue of the 5- or 6-membered heterocyclic ring group where the benzene ring may optionally be further substituted with one or more identical or non-identical substituents selected from the group consisting of straight or branched C₁₋₄ alkyl group, straight or branched C₁₋₄ alkoxy group, trifluoromethyl group, nitro group, C₁₋₂ alkylenedioxy group, amino group, amino group—substituted with one or two identical or non-identical straight or branched C₁₋₄ alkyl group—, halogen atom, and sulfonyl group;
   5-membered heterocyclic ring group containing two or three nitrogen atoms, or one nitrogen atom and one oxygen atom, or one nitrogen atom and one sulphur atom condensed with a 6-membered heteroaromatic ring group containing one or two nitrogen atoms, optionally substituted with one or more identical or non-identical substituents selected from the group consisting of straight or branched C₁₋₄ alkyl group, straight or branched C₁₋₄ alkoxy group, amino group—substituted with one or two, identical or non-identical straight or branched C₁₋₄ alkyl group or benzyl group—, and 1-(C₁₋₄-alkylcarbonyl)-2-phenylethyl group;
      with the proviso that if B stands for SO₂ group and the meanings of Ar¹, X, Y, R¹, R² and Ar² are as defined above, Z means a straight C₁₋₄ alkylene group—optionally substituted with one or more identical or non-identical straight or branched C₁₋₄ alkyl group, and with the further proviso that when Ar¹ represents phenyl group, X means methylene group substituted with one methyl group, Y means propylene group, Z stands for propylene or butylene group, R¹ means methyl group, R² means hydrogen atom and Ar² stands for phenyl group, B is different from —SO₂— group;
      and their salts, solvates and isomers and the salts and solvates thereof.

Especially favourable are the following compounds:
N-{3-[(3,4-Dichlorobenzyl)(methyl)amino]propyl}-2-(5-dimethylaminothiazolo[5,4-b]pyridin-2-ylsulfanyl)acetamide;
N-{3-[(3,4-Dichlorobenzyl)(methyl)amino]propyl}-2-(5-dimethylaminothiazolo[5,4-d]pyrimidin-2-ylsulfanyl)acetamide;
2-(6-Aminobenzoxazol-2-ylsulfanyl)-N-{3-[(3,4-dichlorobenzyl)(methyl)amino]propyl}acetamide;
N-{3-[(3,4-Dichlorobenzyl)(methyl)amino]propyl}-2-(6-methoxybezoxazol-2-ylsulfanyl)acetamide;
N-{3-[(3,4-Dichlorobenzyl)(methyl)amino]propyl}-2-(1,6-dimethyl-1H-benzimidazol-2-ylsulfanyl)acetamide;
N-{3-[(3,4-Dichlorobenzyl)(methyl)amino]propyl}-2-(oxazolo[5,4-b]pyridine-2-ylsulfanyl)acetamide;
2-(6-Aminobenzthiazol-2-ylsulfanyl)-N-{3-[(3,4-dichlorobenzyl)(methyl)amino]propyl}acetamide;
2-(Benzthiazol-2-ylsulfanyl)-N-{3-[(3,4-dichlorobenzyl)(methyl)amino]propyl}acetamide;
N-{3-[(3,4-Dichlorobenzyl)(methyl)amino]propyl}-2-(6-methylbezoxazol-2-ylsulfanyl)acetamide;
N-{3-[(3,4-Dichlorobenzyl)(methyl)amino]propyl}-2-(thiazolo[5,4-b]pyridin-2-ylsulfanyl)acetamide;
2-(Benzoxazol-2-ylsulfanyl)-N-{3-[(3,4-dichlorobenzyl)(methyl)amino]propyl}acetamide;
N-{3-[(3,4-Dichlorobenzyl)(methyl)amino]propyl}-2-(5-methoxybenzothiazol-2-ylsulfanyl)acetamide;
N-{3-[(3,4-Dichlorobenzyl)(methyl)amino]propyl}-2-(6-ethoxybenzothiazol-2-ylsulfanyl)acetamide;
N-{3-[(3,4-Dichlorobenzyl)(methyl)amino]propyl}-2-(5-ethylaminothiazolo[5,4-d]pyrimidin-2-ylsulfanyl)acetamide;
N-{3-[(3,4-Dichlorobenzyl)(methyl)amino]propyl}-2-(5-ethylaminothiazolo[5,4-b]pyridin-2-ylsulfanyl)acetamide;
N-{3-[(3,4-Dichlorobenzyl)(methyl)amino]propyl}-2-(5-isopropylaminothiazolo[5,4-d]pyrimidin-2-ylsulfanyl)acetamide;
N-{3-[(3,4-Dichlorobenzyl)(methyl)amino]propyl}-2-(5-isopropylaminothiazolo[5,4-b]pyridin-2-ylsulfanyl)acetamide;
2-(5-Benzylaminothiazolo[5,4-b]pyridin-2-ylsulfanyl)-N-{3-[(3,4-dichlorobenzyl) (methyl)amino]propyl}acetamide;
2-(6-Aminobenzthiazol-2-ylsulfanyl)-N-{3-[(3,4-dichlorobenzyl)(methyl)amino]butyl}-acetamide;
2-(6-Aminobenzthiazol-2-ylsulfanyl)-N-{3-[(3,4-dichlorobenzyl)(methyl)amino]propyl}-butyramide;
N-{3-[(3,4-Dichlorobenzyl)(methyl)amino]propyl}-2-(6-methyl-1H-benzimidazol-2-ylsulfanyl)acetamide;
N-{3-[(3,4-Dichlorobenzyl)(methyl)amino]propyl}-2-(quinazolin-2-ylsulfanyl)acetamide;
2-(5-Benzylaminothiazolo[5,4-d]pyrimidin-2-ylsulfanyl)-N-{3-[(3,4-dichlorobenzyl) (methyl)amino]propyl}acetamide;
2-(6-Aminobenzthiazol-2-ylsulfanyl)-N-{2-[(3,4-dichlorobenzyl)(methyl)amino]ethyl}-acetamide;
3-(6-Aminobenzthiazol-2-ylsulfanyl)-N-{3-[(3,4-dichlorobenzyl)(methyl)amino]propyl}-propionamide;
2-(6-Aminobenzthiazol-2-ylsulfanyl)-N-{3-[(3,4-dichlorobenzyl)(methyl)amino]propyl}-acetamide;
2-(6-Aminobenzthiazol-2-ylsulfanyl)-N-{3-[(3,4-dichlorophenyl)propyl](methyl)amino]propyl}acetamide;
2-(6-Aminobenzthiazol-2-ylsulfanyl)-N-{3-[(3,4-dichlorobenzyl)(methyl)amino]butyl}-acetamide;
N-{3-[(3,4-Dichlorobenzyl)(methyl)amino]-1-methylpropyl}-2-(6-methylbenzoxazol-2-ylsulfanyl)acetamide;
N-{3-[(3,4-Dichlorobenzyl)(methyl)amino]-1-methylpropyl}-2-(thiazolo[5,4-b]pyridin-2-ylsulfanyl)acetamide;
2-(6-Aminobenzthiazol-2-ylsulfanyl)-N-{3-[(3,4-dichlorobenzyl)(methyl)amino]-2-methylpropyl}acetamide;
2-(6-Aminobenzthiazol-2-ylsulfanyl)-N-{3-[(3,4-dichlorobenzyl)(methyl)amino]-1-methylpropyl}acetamide;
2-(6-Aminobenzthiazol-2-ylsulfanyl)-N-{3-[(3,4-dichlorobenzyl)(methyl)amino]propyl}-N-methylacetamide;
(+)N-{3-[(3,4-Dichlorobenzyl)(methyl)amino]-1-methylpropyl}-2-(6-methylbenzoxazol-2-ylsulfanyl)acetamide;
(−)N-{3-[(3,4-Dichlorobenzyl)(methyl)amino]-1-methylpropyl}-2-(6-methylbenzoxazol-2-ylsulfanyl)acetamide;
2-(6-Aminobenzthiazol-2-ylsulfanyl)-N-{3-[(3,4-dichlorobenzyl)(methyl)amino]propyl}propionamide; and
N-{3-[(3,4-Dichlorobenzyl)(methyl)amino]propyl}-2-(4-methylbenzoxazol-2-yl)sulfinyl]acetamide; and their salts, solvates, isomers and the salts and solvates thereof.

The present invention relates furthermore to the pharmaceutical preparations containing the compounds of the general formula (I) or its isomers, salts or solvates, which are preferably oral preparations, but inhalable, parenteral and transdermal preparations also form a subject of the present invention. The above pharmaceutical preparations may be solid or liquid formulations, for example tablets, pellets, capsules, patches, solutions, suspensions or emulsions. The solid formulations, first of all the tablets and capsules are preferred.

The above pharmaceutical preparations are prepared by applying the usual excipients and technological operations.

The compounds of the general formula (I) according to the invention can be used for the treatment of pathologies where CCR3 receptors play a role in the development of the disease.

The compounds according to the present invention can favourably used in the treatment of diseases selected from asthma, allergic rhinitis, atopic dermatitis, eczema, inflammatory bowel disease, ulcerative colitis, allergic conjunctivitis, multiple sclerosis, Crohn's disease, HIV-infection and diseases in conjunction with AIDS.

A further subject of the invention is the use of the compounds of the general formula (I) for the treatment of the above pathologies. The suggested daily dose is 1-100 mg of the active component, depending on the nature and severity of the disease and the sex and weight of the patient.

The invention relates furthermore to the preparation of the compounds of the general formula (I)—where in the formula the meanings of B, Ar¹, X, Y, Z, R¹, R² and Ar² are as defined above—and their salts, solvates and isomers.

The compounds of the general formula (III), applied in the process according to the invention, are new and they also form a subject of the invention. The meanings of the substituents of general formula (III) are as defined above, Hal stands for halogen atom.

Scheme 1. presents one possible method for the preparation of the compounds of general formula (I) (process version a.).

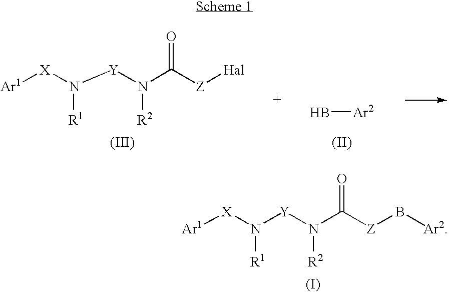

In process version a.) according to the invention a halogen compound of general formula (III),

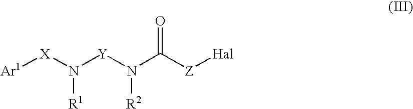

where Ar¹, X, Y, Z, R¹ and R² have the same meaning as above and Hal stands for halogen atom, is reacted with a compound of general formula (II),

HB—Ar² (II)

where the meanings of Ar² and B are as defined above and, if desired the substituents of the compound of general formula (I) thus obtained are transformed into each other by using known methods and/or the resulting compound of general formula (I) is transformed into its salt or solvate, or liberated from its salt or solvate and/or resolved into its optically active isomers, or the optically active isomer is transformed into the racemic compound and if desired the structural isomers are separated from each other.

In the compound of general formula (III) the meaning of Hal is favourably bromo or chloro atom.

The reaction according to process version a.) is performed preferably in inert solvent for example in dichloromethane, chloroform, tetrahydrofuran, acetonitrile or in the mixture of thereof, preferably in N,N-dimethylformamide, in the presence of organic bases, as for example triethylamine, diethyl-i-propylamine, or inorganic bases, preferably potassium carbonate at a temperature between 0° C.-100° C., preferably at room temperature.

Scheme 2. presents another possible route for the preparation of the compounds of general formula (I) (process version b.).

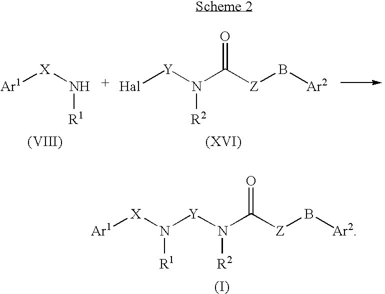

In process version b.) according to the invention an amine of general formula (VIII),

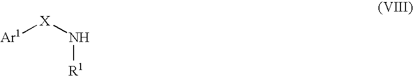

where the meanings of Ar¹, X and R¹ are as defined above, is reacted with a halogen compound of general formula (XVI),

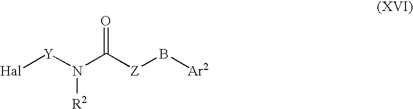

where the meanings of Y, R², Z, B and Ar² are as defined above and Hal stands for halogen atom, and if desired, the substituents of the compound of general formula (I) thus obtained are transformed into each other by using known methods and/or the resulting compound of general formula (I) is transformed into its salt or solvate, or liberated from its salt or solvate and/or resolved into its optically active isomers, or the optically active isomer is transformed into the racemic compound and if desired the structural isomers are separated from each other.

The reaction of the amine of general formula (VIII) and the halogen compound of general formula (XVI) is performed in an inert solvent, preferably in dichloromethane, in the presence of organic bases as acid binders.

Scheme 3. presents a third possible route for the preparation of the compounds of general formula (I) (process version c.).

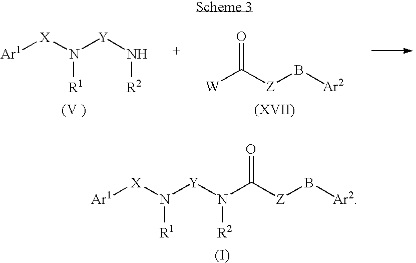

In process version c.) according to the invention a diamine of general formula (V),

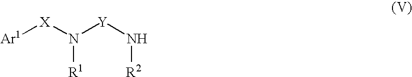

where the meanings of Ar¹, X Y, R¹ and R² are as defined above, is reacted with a carboxylic acid derivative of general formula (XVII),

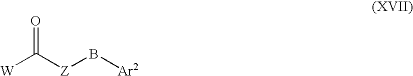

where the meanings of Ar², Z and B are as defined above and W stands for halogen atom, hydroxyl group, —OR¹¹-group, wherein R¹¹ means C₁₋₄-alkyl group or —O—CO-Z-B—Ar²-group, wherein the meaning of Z, B and Ar² are as defined above, and if desired, the substituents of the compound of general formula (I) thus obtained are transformed into each other by using known methods and/or the resulting compound of general formula (I) is transformed into its salt or solvate, or liberated from its salt or solvate and/or resolved into its optically active isomers, or the optically active isomer is transformed into the racemic compound and if desired the structural isomers are separated from each other.

In a preferred embodiment of process c.) according to the invention, the acid of general formula (XVII)—where W stands for hydroxyl group—is transformed into an acid chloride, by using acid chloride-forming reagents, favourably thionyl chloride, and the resulting acid chloride is reacted in an inert solvent, like dichloromethane, chloroform, or ethyl acetate, with the amine of general formula (V), in the presence of a base, like triethylamine, or in pyridine, or in aqueous alkali solution, at room temperature or under reflux conditions.

In another preferred method the acid of general formula (XVII)—where W stands for hydroxyl group—is reacted with the amine of general formula (V), in the presence of an activating agent. Activation of the carboxylic acid may take place via mixed anhydride intermediates, by using e.g pivalyl chloride (M. T. Leplawy: Tetrahedron 1960, 11, 39), ethyl chloroformate (T. Wieland: J. Liebigs Ann. Chem. 1951, 572, 190), isobutyl chloroformate (J. R. Vaughan: JACS. 1951, 73, 3547) or dicyclohexylcarbodiimide (DCC) (R. Arshady: J. Chem. Soc. Perkin Trans. 1, 1981, 529 or D. Hudson: J. Org. Chem. 1988, 53, 617) in an inert solvent, e.g. in dichloromethane, chloroform, tetrahydrofuran, acetonitrile, in the presence of an acid binding tertiary amine, e.g. triethylamine, N-methylmorpholine, at a temperature of −10° C. to 25° C.

The activation can furthermore be accomplished by use of carbonyldiimidazole (H. A. Staab: Lieb. Ann. Chem.: 1957, 609, 75), in an inert solvent, preferably in dichloromethane, chloroform, tetrahydrofuran, acetonitrile or in the mixture thereof or with benzotriazol-1-yl-oxy-tripyrrolidinophosphonium hexafluorophosphate (PyBOP), in an inert solvent (J. Corte: Tetrahedron Lett. 31, 1990, 205).

If the compound of the general formula (XVII) is a carboxylic acid ester, where in the formula W means an OR¹¹-group, the reaction can be carried out by one of the methods known in the literature, preferably at 100° C.-150° C., without solvent, in melt.

If the compound of the general formula (I) is a racemic compound, the separation of the enantiomers can be accomplished by chiral preparative column chromatography or by another known method suitable for the resolution of compounds of basic character.

The compounds of the general formula (II) are in part known in the literature, or they can be prepared by a method known in the literature (e.g. WO 02/066035, James A. T. and co-workers: J. Chem. Soc. 1950, 1515-1519; Chu-Moyer and co-workers: J. Org. Chem. 1995, 60, 17, 5721-5725; Garin J. and co-workers: Synthesis 1985, 9, 867-870; Haviv F. and co-workers: J. Med. Chem. 1988, 31, 9, 1719-1728) or they are commercially available.

Scheme 4. presents the preparation of the compounds of general formula (III).

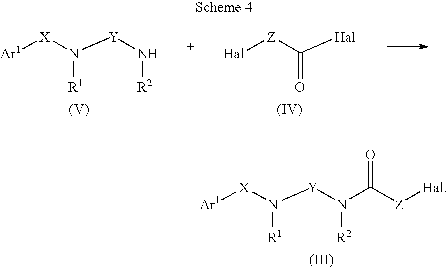

The halogen compounds of general formula (III)—where in the formula the meanings of Ar¹, X, R¹, Y, R² and Z are as defined above and Hal stands for halogen atom, preferably chloro or bromo atom—are new compounds, they can be prepared by known methods (e.g. Chem. Pharm. Bull. 2003, 51, 6, 697-701; J. Chem. Soc. Perkin Transl. 1993, 2, 613; JACS. 1947, 69, 515; J. Med. Chem. 1998, 41, 11, 1943) from the diamines of general formula (V)—where in the formula the meanings of Ar¹, X, R¹, Y, and R² are as defined above—with the acyl bromides or acyl chlorides of general formula (IV)—where in the formula the meaning of Z is as defined above—by methods known in the literature, in inert solvents, for example in dichloromethane, tetrahydrofuran or acetonitrile or in the mixture thereof, preferably in dichloromethane at room temperature or at lower temperatures.

The diamines of general formula (V) can be prepared by different methods depending on the nature of the substituents R¹, R², X and Y.

Scheme 5. presents the preparation of those compounds belonging to general formula (V) where in the formula R² stands for hydrogen atom, Y stands for 1,3-propylene, 1-methyl-1,3-propylene, 2-methyl-1,3-propylene or 1,4-butylene (R⁶ and R⁷ independently from each other represents hydrogen atom or methyl group, p is 0 or 1), and the meanings of Ar¹ and X are as defined above.

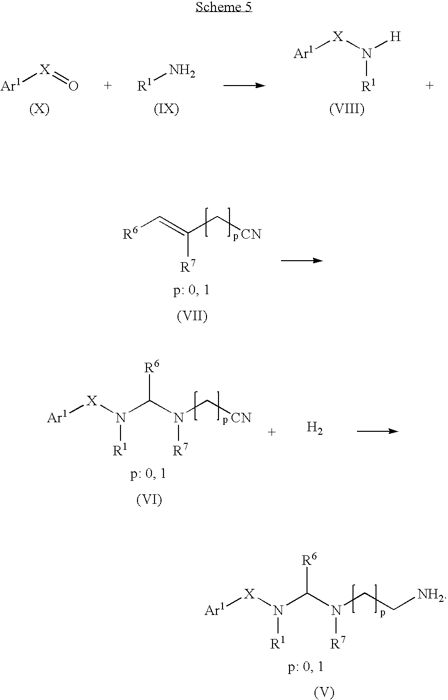

The compounds of the general formula (VIII) can be prepared by methods known in the literature starting from the oxo compounds (aldehydes or ketones) of the general formula (X) by reductive amination with the amines of general formula (IX) in alcoholic medium, in the presence of sodium cyanoborohydride (Holzgrabe U.: Arch. Pharm. 1987, 320, 7, 647-654), or by catalytic hydrogenation (Elslager E. F.: J. Med. Chem. 1981, 24, 2, 140-145), or with sodium borohydride in aqueous alcohol medium (Simig Gy.: J. Chem. Soc Perkin Trans. 1. 1992, 13, 1613-16). The compounds of the general formula (IX) are commercially available. The aldehydes of general formula (X) are commercially available or can be prepared by methods known in the literature. The compounds of general formula (VI) can be prepared from the compounds of general formula (VIII) with the alkene-cyanides of the general formula (VII) by literature analogies (King M. et al: JACS. 1946, 68, 1468, or Surrey et al: JACS. 1956, 78, 2573). The cyanides of the general formula (VII) are commercially available. The diamines of the general formula (V) can be obtained by catalytic hydrogenation of the cyanides of general formula (VI) by literature analogies, in alcohol or hexane solution, in the presence of ammonia and Raney nickel or rhodium catalyst, in a given case under pressure (Shapiro et al: JACS. 1959, 81, 3083-84, and Roufos I.: J. Med. Chem. 1996, 39, 7, 1514).

The diamines of the general formula (V), where in the formula the meaning of Y is ethylene group, R² stands for hydrogen atom and the meanings of Ar¹ and X are as defined above, can be prepared as shown in Scheme 6.,

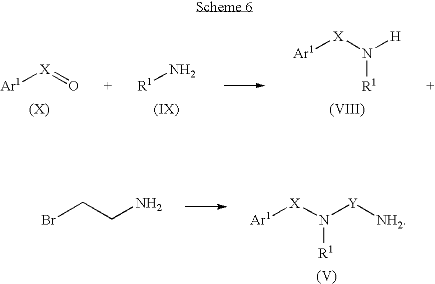

from the amines of the general formula (VIII) with 2-bromoethylamine, by literature analogy, in hot aqueous solution (Arz. Forsch. 1975, 25, 1853-58).

The diamines of the general formula (V), where R² stands for hydrogen atom, Y for 3-methylpropylene group and the meanings of Ar¹ and X are as defined above, can be prepared as shown in Scheme 7.

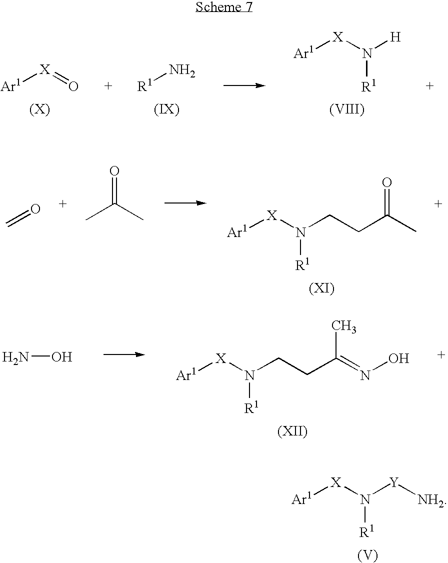

The compounds of general formula (XI) are obtained by Mannich condensation from the amines of general formula (VIII) with paraformaldehyde and acetone. By literature analogy, the reaction can be performed in i-propanol under reflux conditions (JACS. 1959, 81, 2214-18). The oximes of general formula (XII) are prepared from the compounds of general formula (XI) with hydroxylamine, by literature analogies, in aqueous i-propanol solution (JACS. 1959, 81, 2214-18). The amine of general formula (V) is prepared by literature analogy from the oxime of general formula (XII) by catalytic hydrogenation in the presence of Raney-Nickel catalyst, in ethanolic ammonia solution.

Scheme 8. demonstrates the preparation of the compounds of general formula (V) where R¹ and R² represents methyl group and the meanings of Ar¹, X and Y are as defined above.

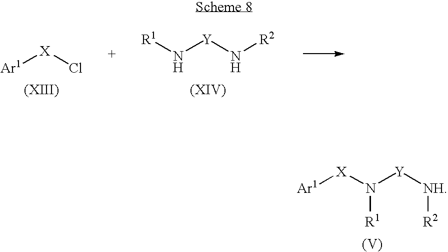

The compounds of the general formula (V) can be obtained by reacting the commercially available halogenides of the general formula (XIII) with the N,N′-dimethylaminoalkyl compounds of general formula (XIV), in inert solvents, preferably in acetonitrile, in the presence of an acid binding organic amine.

The compounds of the general formula (X), where X represents 1,3-propylene group and the meaning of Ar¹ is as defined above, can be obtained as presented in Scheme

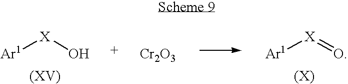

by analogies in the literature (J. Org. Chem. 2002, 67, 25, 8758-8763), from the appropriate alcohols of general formula (XV) by oxidation with pyridinium chlorochromate in inert solvent, preferably in dichloromethane.

The ketones of general formula (X), where X represents 3-methylpropylene group, can be prepared by the method shown in Scheme 10.,

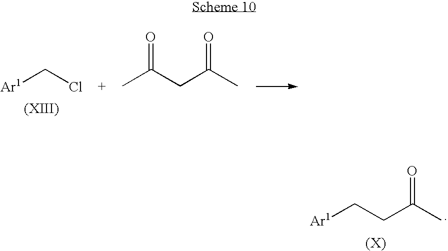

by analogies in the literature (Powel et al: JACS. 2004, 126, 25, 7788-89), by heating the commercially available benzylchlorides of general formula (XIII) with pentane-2,4-dione in alcohol solution under reflux conditions, in the presence of potassium carbonate.

The intermediate (XVI) can be prepared by the method shown in Scheme 11., by analogy of the above process version c.), used for the preparation of compounds of general formula (I) of the invention.

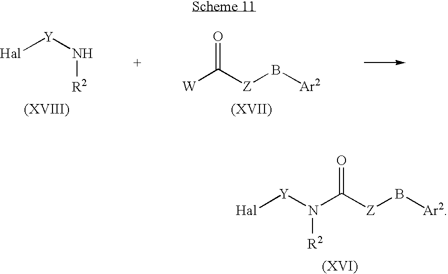

One possible method to obtain the acid derivative of general formula (XVII) where the meanings of W, Z, B and Ar² are as defined above, is presented in Scheme 12.

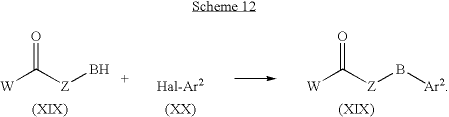

The acid derivative of general formula (XIX) containing the appropriate BH-group can be reacted with the halogenide of general formula (XX), in an inert solvent, preferably in dichloromethane in the presence of an organic base, preferably triethylamine or 4-methylmorpholine or, in another method, in inert solvent, preferably tetrahydrofuran, in the presence of sodium hydride.

Further details of invention are demonstrated by the examples, without limiting the invention to the examples.

### EXAMPLES

### Example 1

### N-{3-[(3,4-Dichlorobenzyl)(methyl)amino]propyl}-2-(5-dimethylaminothiazolo[5,4-b]-pyridin-2-ylsulfanyl)acetamide (I)

In the general formula (I) Ar¹ stands for 3,4-dichlorophenyl group, X and Z for methylene group, R¹ for methyl group, Y for 1,3-propylene group, R² for hydrogen atom, B for sulfur atom, Ar² for 5-dimethylaminothiazolo[5,4-b]pyridin-2-yl group.

### a.) N-(3,4-Dichlorobenzyl)methylamine hydrogen chloride salt (VIII)

(Simig Gy.: J. Chem. Soc. Perkin Trans. I. 1992, 13, 1613-16)

17.5 g (100 mmol) 3,4-dichlorobenzaldehyde is dissolved in 40 ml methanol and under stirring 15.6 ml 40% aqueous methylamine (200 mmol) in 30 ml methanol is added to it. The reaction mixture is cooled to 0° C. and in small portions 1.9 g (50 mmol) sodium borohydride is added, while keeping the temperature at 0° C.

Without cooling-bath the reaction mixture is allowed to reach room temperature and stirring is continued for 28 hours. Methanol is distilled off in vacuo and to the residue 200 ml dichloromethane was added. The mixture is extracted with 3×50 ml water, the organic phase is dried over sodium sulfate and evaporated in vacuo. The crude product is dissolved in 100 ml ethyl acetate and acidified with hydrogen chloride saturated solution in ether (50 ml.) The resulting crystals are filtered off, washed consecutively with ethyl acetate and ether to obtain 20 g of the title compound as white crystals.

Mp: 225° C.

### b.) 3-[(3,4-Dichlorobenzyl)(methyl)amino]propionitrile (VI)

From 20 g (88 mmol) N-(3,4-Dichlorobenzyl)methylamine hydrogen chloride salt the base is liberated by the addition of 12.6 ml (90 mmol) triethylamine in 100 ml ethyl acetate solution. The resulting 16.5 g base is dissolved in 170 ml abs. methanol, the solution is cooled to below 0° C. and 5.7 ml (87 mmol) acrylonitrile is added to it. The reaction mixture is stirred at 0° C. for 30 minutes, allowed to reach room temperature, stirred for 30 hours and evaporated to obtain 20 g of the title compound in the form of an oil.

LC/MS[MH⁺]=243 (C₁₁H₁₂Cl₂N₂ 243.14).

### c.) N-(3,4-Dichlorobenzyl)-N-(methyl)propan-1,3-diamine (V)

20 g (82.3 mmol) 3-[(3,4-Dichlorobenzyl)(methyl)amino]propionitrile is hydrogenated at room temperature, in the presence of Raney-Nickel catalyst, in ethanolic ammonia solution in (100 ml). After removal of the solvent 20 g title compound is obtained in the form of an oil. LC/MS[MH⁺]=247 (C₁₁H₁₆Cl₂N₂ 247.17)

### d.) 2-Bromo-N-{3-[(3,4-dichlorobenzyl)(methyl)amino]propyl}acetamide hydrogen bromide salt (III)

4.9 g (20 mmol) N-(3,4-Dichlorobenzyl)-N-(methyl)propan-1,3-diamine is dissolved in 50 ml dichloromethane. The solution is cooled to −10° C. and at that temperature 2 ml (23 mmol) bromoacetyl bromide in 12 ml dichloromethane is added to it dropwise. The reaction mixture is stirred at −10° C. for 10 minutes and at room temperature for 3 hours. Dichloromethane is poured off, the residue is stirred with 15 ml abs. ethanol, the precipitated crystals are filtered off, washed with ethanol and with ether to obtain 7 g title compound in the form of its hydrogen bromide salt. Mp.: 141° C.

### e.) N-{3-[(3,4-Dichlorobenzyl)(methyl)amino]propyl}-2-(5-dimethylamino thiazolo[5,4-b]pyridin-2-ylsulfanyl)acetamide (I)

To the solution of 0.5 g (2.4 mmol) 5-dimethylaminothiazolo[5,4-b]pyridin-2-thiol (II) in 15 ml dimethylformamide are added 0.7 g (5 mmol) potassium carbonate, then 1.1 g (2.4 mmol) 2-bromo-N-{3-[(3,4-dichlorobenzyl)(methyl)amino]propyl}acetamide hydrogen bromide salt (III) in 10 ml dimethylformamide. The reaction mixture is stirred for 3 hours, then poured onto ice-water. The mixture is extracted with ethyl acetate, the organic phase is dried over sodium sulfate, evaporated, the residue is mixed with ether, the solid material is filtered off to obtain 0.88 g title compound.

Mp.: 92-93° C.

### Examples 2-74

The compounds of Table 1. are prepared according to the procedures described in Example 1.

TABLE 1

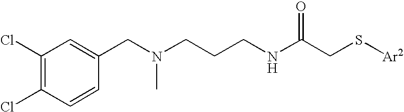

Ar¹ = 3,4-dichlorophenyl
X = —CH₂—
R¹ = —CH₃
Y = —CH₂—CH₂—CH₂—
R² = H
Z = CH₂

| Ex- | | | |
|---|---|---|---|
| am- | | Mp | |
| ple | Ar² | (° C.) | [MH⁺] |
| 2. | 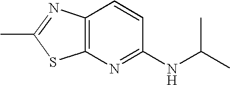 | | 540 |
| 3. | 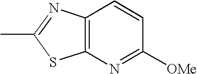 | 65-66 | |
| 4. | 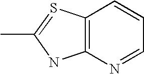 | 118-120 | |
| 5. | 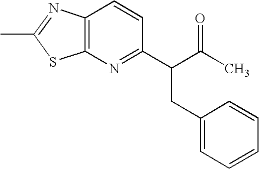 | 75-77 | |
| 6. | 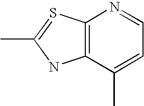 | | 469 |
| 7. | 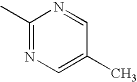 | 76-78 | |
| 8. | 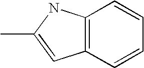 | 176-185 | |
| 9. | 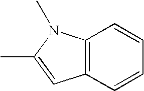 | 88-88.5 | |
| 10. | 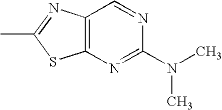 | 142-145 | |
| 11. | 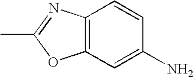 | 100-101 | |
| 12. | 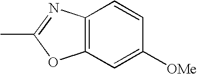 | 52-53 | |
| 13. | 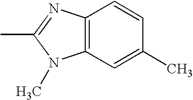 | | 465 |
| 14. | 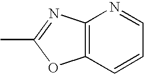 | 80-81.5 | |
| 15. | 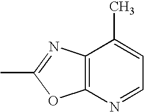 | | 453 |
| 16. | 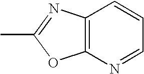 | | 439 |
| 17. | 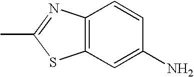 | 88-90.5 | |
| 18. | 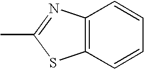 | | 454 |
| 19. | 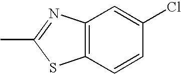 | | 488 |
| 20. | 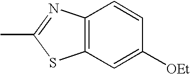 | 68-70 | |
| 21. | 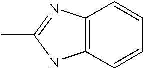 | 114-115 | |
| 22. | 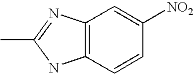 | | 482 |
| 23. | 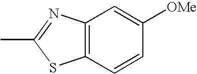 | | 484 |
| 24. | 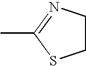 | | 406 |
| 25. | 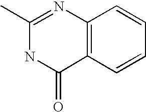 | 203 | |
| 26. | 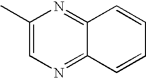 | 64-66 | |
| 27. | 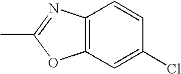 | 82-84 | |
| 28. | 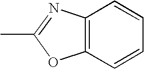 | | 438 |
| 29. | 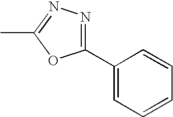 | 91-92 | |
| 30. | 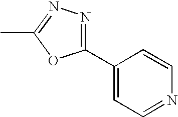 | 102-104 | |
| 31. | 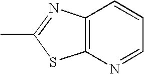 | | 455 |
| 32. | 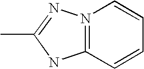 | | 438 |
| 33. | 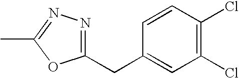 | | 547 |
| 34. | 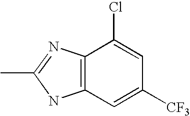 | | 539 |
| 35. | 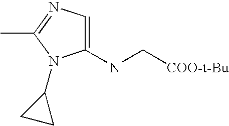 | | 557 |
| 36. | 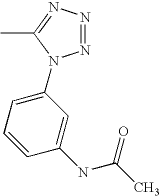 | | 522 |
| 37. | 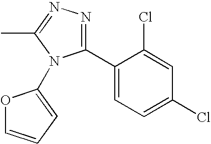 | | 612 |
| 38. | 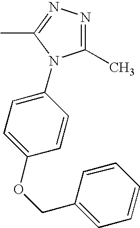 | | 584 |
| 39. | 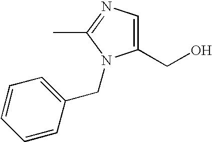 | | 507 |
| 40. | 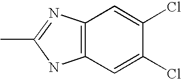 | 155 | |
| 41. | 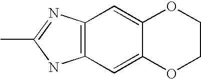 | 114-116 | |
| 42. | 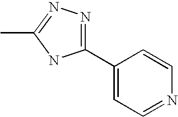 | 112-115 | |
| 43. | 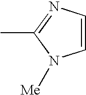 | | 401 |
| 44. | 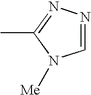 | | 402 |
| 45. | 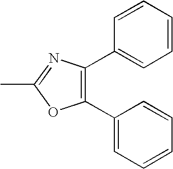 | | 540 |
| 46. | 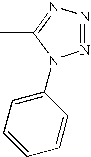 | | 465 |
| 47. | 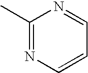 | 51-53 | |
| 48. | 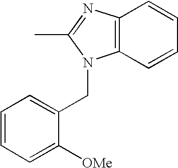 | | 557 |
| 49. | 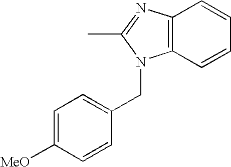 | | 557 |
| 50. | 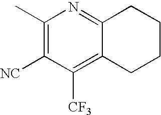 | 90-91 | |
| 51. | 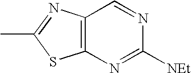 | 77-79 | |
| 52. | 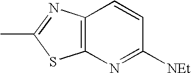 | | 498 |
| 53. | 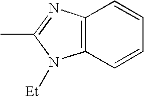 | | 465 |
| 54. | 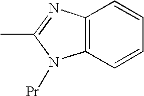 | | 479 |
| 55. | 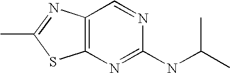 | 108-110 | |
| 56. | 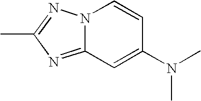 | | 463 |
| 57. | 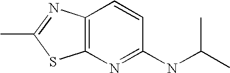 | 113-115 | |
| 58. | 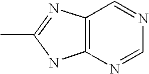 | 115-117 | |
| 60. | 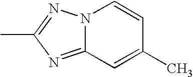 | 82-83 | |
| 61. | 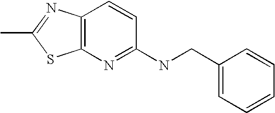 | | 560 |
| 62. | 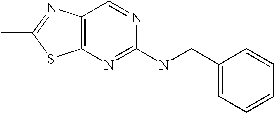 | 110-112 | |
| 63. | 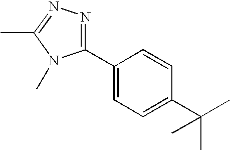 | 151 | |
| 64. | 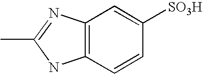 | | 516 |
| 65. | 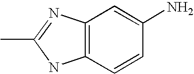 | | 452 |
| 66. | 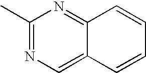 | 83-85 | |
| 67. | 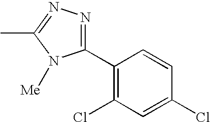 | 85-86 | |
| 68. | 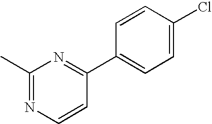 | 85-8 | |
| 69. | 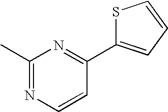 | | 481 |
| 70. | 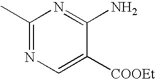 | 79-81 | |
| 71. | 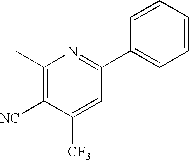 | 143-145 | |
| 72. | 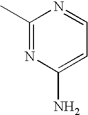 | 92-94 | |
| 73. | 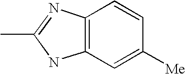 | 112-113 | |
| 74. | 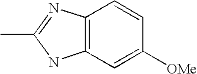 | 97-98 | |

### Example 75

### N-{3-[(3,4-Dichlorobenzyl)(methyl)amino]propyl}-2-(1-methyl-1H-benzimidazol-2-ylsulfanyl)acetamide

In the general formula (I) Ar¹ stands for 3,4-dichlorophenyl group, X and Z for methylene group, R¹ for methyl group, Y for 1,3-propylene group, R² for hydrogen atom, B for sulfur atom, Ar² for 1-methylbenzimidazol-2-yl-group.

### a.) 2-Chloro-1-methyl-1H-benzimidazol

(Galy J-P. Et al.: J. Het. Chem. 1997, 34, 6, 1781-88)

To the solution of 3 g (20 mmol) 2-chlorobenzimidazole in 30 ml water under cooling on ice-bath 9 ml 5 N sodium hydroxide solution and then 3.3 ml (34.7 mmol) dimethyl sulfate is added. The reaction mixture is stirred at room temperature for 2 hours, the precipitated crystals are filtered off, washed with water and dried to obtain 2.8 g title compound.

Mp: 115-117° C.

### b.) Methyl-(1-methyl-1H-benzimidazol-2-ylsulfanyl)acetate

To the solution of 1.16 g (11 mmol) thioglycolic acid methyl ester in 14 ml chloroform 1.2 g (12 mmol) triethylamine and the solution of 1.33 g (8 mmol) 2-chloro-1-methyl-1H-benzimidazol in 10 ml chloroform are added. The reaction mixture is heated at 60° C. for 20 hours. The chloroform solution is washed with water, with diluted potassium hydrogen sulfate solution and with water, dried over sodium sulfate and evaporated. The residue is purified by column chromatography using hexane-ethyl acetate 2:1 mixture as eluent. The precipitated crystals are filtered off. 0.52 g title compound is obtained. LC/MS[MH⁺]=237 (C₁₁H₁₂N₂O₂S 236.29)

### c.) N-{3-[(3,4-Dichlorobenzyl)(methyl)amino]propyl}-2-(1-methyl-1H-benzimidazol-2-ylsulfanyl)acetamide

The mixture of 0.52 g (2.2 mmol) methyl (1-methyl-1H-benzimidazol-2-ylsulfanyl)acetate and 0.61 g (2.5 mmol) N-(3,4-dichlorobenzyl)-N-(methyl)propan-1,3-diamine is heated at 100° C. for 1 hour. The melt is purified by column chromatography using chloroform as eluent. 350 mg title compound is obtained in the form of an oil. LC/MS[MH⁺]=451 (C₂₁H₂₄Cl₂N₄OS 451.41)

### Example 76

### N-{3-[(3,4-Dichlorobenzyl)(methyl)amino]propyl}-2-(6-methylbenzoxazol-2-ylsulfanyl)acetamide

In the general formula (I) Ar¹ stands for 3,4-dichlorophenyl group, X and Z for methylene group, R¹ for methyl group, Y for 1,3-propylene group, R² for hydrogen atom, B for sulfur atom, Ar² for 6-methylbenzoxazol-2-yl-group.

### a.) 6-Methylbenzoxazole-2-thiol

(Haviv F. et al.: J. Med. Chem. 1988, 31, 9, 1719-28)

3.7 g (30 mmol) 2-hydroxy-4-methylaniline is suspended in 50 ml ethanol, 4.8 g (30 mmol) O-ethyl-xanthic acid potassium salt is added to it and the mixture is heated under reflux conditions for 16 hours. The solvent is removed, the residue is dissolved in water, acidified with acetic acid to pH 5, the precipitated crystals are filtered off, washed with water. 4.3 g title compound is obtained. Mp: 209° C.

### b.) 2-Chloro-6-methylbenzoxazole

(Haviv F. et al.: J. Med. Chem. 1988, 31, 9, 1719-28)

4.13 g (25 mmol) 5-methylbenzoxazol-2-thiol is suspended in 40 ml toluene, slowly 6.2 g (30 mmol) phosphor pentachloride is added to it and the mixture is heated under reflux conditions for 16 hours. The solvent is removed, to the residue ether is added, the precipitated inorganic salts are filtered off, the ether solution is evaporated. 2.8 g title compound is obtained in the form of an oil. LC/MS[MH⁺]=168 (C₈H₆ClNO 167.594).

### c.) Methyl-(6-methylbenzoxazol-2-ylsulfanyl)acetate

0.27 g (2.6 mmol) thioglycolic acid methyl ester is dissolved in 8 ml tetrahydrofuran, 0.132 g (3.3 mmol) 60% sodium hydride is added, the mixture is stirred at room temperature for 15 minutes, then the solution of 0.4 g (2.4 mmol) 2-chloro-6-methylbenzoxazole in 20 ml tetrahydrofuran is added to it. The reaction mixture is stirred at 50° C. for 3 hours, the solvent is removed, the residue is extracted with water and ethyl acetate, the organic phase is dried over sodium sulfate and evaporated to obtain the title compound which is carried into the next step without purification. LC/MS[MH⁺]=238 (C₁₁H₁₁NO₃S 237.278).

### d.) (6-Methylbenzoxazol-2-ylsulfanyl)acetic acid

To 0.57 g (2.4 mmol) methyl (6-methylbenzoxazol-2-ylsulfanyl)acetate, 10 ml methanol and 4.8 ml 2N sodium hydroxide solution are added and the mixture is stirred at room temperature for 12 hours. The solvent is removed, to the residue water is added and the mixture is acidified with potassium hydrogen sulfate. The precipitated crystals are filtered off, washed with water. 0.34 g title compound as white crystals are obtained. Mp: 144-146° C.

### e.) N-{3-[(3,4-Dichlorobenzyl)(methyl)amino]propyl}-2-(6-methylbenzoxazol-2-ylsulfanyl)acetamide

To the solution of 0.33 g (1.5 mmol) (6-methylbenzoxazol-2-ylsulfanyl)acetic acid in 10 ml chloroform 0.15 g (1.5 mmol) N-methylmorpholine is added. The mixture is cooled to −10° C., 0.2 g (1.5 mmol) tert-butyl chloroformate is added to it and stirred for 15 minutes. Then 0.42 g (1.7 mM) N-(3,4-dichlorobenzyl)-N-(methyl)propane-1,3-diamine in 3 ml chloroform is added to it and the mixture is stirred for 30 minutes under cooling and 30 minutes at room temperature. The chloroform solution is washed with water and with 5% potassium hydrogen sulfate solution, dried over sodium sulfate and evaporated in vacuum. The resulting oil is purified by column chromatography to obtain 230 mg title compound in the form of an oil. LC-MS[MH⁺]=452 (C₂₁H₂₃Cl₂N₃O₂S 452.404).

### Example 77

### N-{3-[(3,4-Dichlorobenzyl)(methyl)amino]propyl}-2-(4-methylbenzoxazol-2-ylsulfanyl)acetamide oxalate

In the general formula (I) Ar¹ stands for 3,4-dichlorophenyl group, X and Z for methylene group, R¹ for methyl group, Y for 1,3-propylene group, R² for hydrogen atom, B for sulfur atom, Ar² for 4-methylbenzoxazol-2-yl-group.

The procedure as described in Example 76. is followed starting from 0.44 g (2 mmol) (4-methylbenzoxazol-2-ylsulfanyl)acetic acid and the oxalate salt is formed from the product. Thus, 800 mg title compound is obtained in the form of white crystals. Mp: 149-150° C.

### Example 78

### N-{3-[(3,4-Dichlorobenzyl)(methyl)amino]propyl}phenylsulfanyl)acetamide

In the general formula (I) Ar¹ stands for 3,4-dichlorophenyl group, X and Z for methylene group, R¹ for methyl group, Y for 1,3-propylene group, R² for hydrogen atom, B for sulphur atom, Ar² for phenyl group.

### a.) N-(3-Bromopropyl)(phenylsulfanyl)acetamide

0.44 g (2 mmol) 3-bromopropylamine hydrogen bromide is dissolved in the solution of 0.16 g (4 mmol) sodium hydroxide in 4 ml water and under cooling on ice-water bath, 0.37 g (2 mmol) phenylsulfanylacetyl chloride is added to it. The reaction mixture is stirred for 1 hour under cooling and for 5 hours at room temperature. The precipitated crystals are filtered off and washed with water to obtain the title compound.

LC-MS[MH⁺]=289 (C₁₁H₁₄BrNOS 288.21).

### b.) N-{3-[(3,4-Dichlorobenzyl)(methyl)amino]propyl}phenylsulfanyl)acetamide

To the solution of 0.28 g (1.5 mmol) (3,4-dichlorobenzyl)(methyl)amine in 3 ml dichloromethane 0.2 ml (1.5 mmol) triethylamine is added, then 0.43 g (1.5 mmol) N-(3-bromopropyl)(phenylsulfanyl)acetamide in 3 ml dichloromethane is added dropwise and the mixture is stirred at room temperature for 4 hours. After removal of the solvent water and ethyl acetate are added and the mixture is extracted with 3×15 ml ethyl acetate. The organic phase is washed with water, dried over sodium sulfate and evaporated in vacuum to obtain the title compound. LC-MS[MH⁺]=397 (C₁₉H₂₂C₁₂N₂OS 397.37).

### Examples 79-81

The compounds of Table 2. are prepared according to the procedure as described in Example 1.

TABLE 2

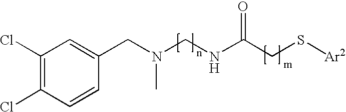

Ar¹ = 3,4-dichlorophenyl
X = —CH₂—
R¹ = —CH₃
Y = —(CH₂)ₙ—
R² = H
Z = —(CH₂)ₘ—

| | | | | Mp | |
|---|---|---|---|---|---|
| Example | n | m | Ar² | (° C.) | [MH⁺] |
| 79. | 3 | 3 | 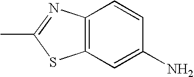 | | 497 |
| 80. | 4 | 1 | 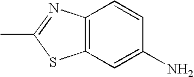 | | 483 |
| 81. | 3 | 2 | 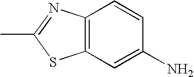 | 87-89 | |

### Examples 82-85

The compounds of Table 3. are prepared according to the procedure as described in Example 1.

TABLE 3

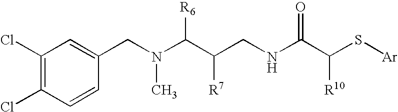

Ar¹ = 3,4-dichlorophenyl
X = —CH₂—
R¹ = —CH₃
Y = —CH(R⁶)—CH(R⁷)—CH₂—
R² = H
Z = CH(R¹⁰)—

| | | | | | Mp | |
|---|---|---|---|---|---|---|
| Example | [symbol] | R⁷ | R¹⁰ | Ar² | (° C.) | [MH⁺] |
| 82. | Me | H | H | 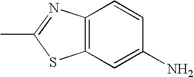 | | 483 |
| 83. | H | Me | H | 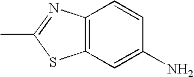 | 91-93 | |
| 84. | H | H | 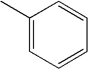 | 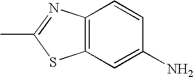 | | 545 |
| 85. | H | H | Me | 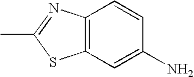 | | 483 |

### Example 86

### 2-(6-Aminobenzothiazol-2-yl-sulfanyl)-N-{2-[(3,4-dichlorobenzyl)(methyl)amino]ethyl}acetamide (I)

In the general formula (I) Ar¹ stands for 3,4-dichlorophenyl group, X and Z for methylene group, R¹ for methyl group, Y for ethylene group, R² for hydrogen atom, B for sulphur atom, Ar² for 6-aminobenzothiazol-2-yl group.

### c.) N-(3,4-Dichlorobenzyl)]-N-(methyl)ethane-1,2-diamine

The N-(3,4-dichlorobenzyl)methylamine (VIII) (4.8 g, 25.5 mmol) prepared according to Example 1.a.) is dissolved in 4 ml water and heated to 95° C. To this mixture is added dropwise the solution of 1.7 g (8.5 mmol) 2-bromomethylamine hydrogen bromide salt in 3 ml water. The reaction mixture is heated for 2 hours, then after cooling to room temperature it is saturated with solid sodium hydroxide. The aqueous solution is extracted with 3×10 ml ether, dried over sodium sulfate, evaporated in vacuum and purified by column chromatography using chloroform-methanol 2:1 mixture as eluent. 1.9 g title compound is obtained in the form of an oil. LC/MS[MH⁺]=233 (C₁₀H₁₄N₂Cl₂ 233.14).

### d.) 2-Bromo-N-{2-[(3,4-dichlorobenzyl)(methyl)amino]ethyl}acetamide hydrogen bromide salt

The N-(3,4-dichlorobenzyl)]-N-(methyl)ethane-1,2-diamine (1 g, 4.3 mmol) of point c.) is treated with 0.94 g (4.7 mmol) bromoacetyl bromide similarity as described in Example 1.d.) to obtain 1.45 g of the title compound. Mp.: 162-165° C.

### e.) 2-(6-Aminobenzothiazol-2-ylsulfanyl)-N-{2-[(3,4-dichlorobenzyl)(methyl)amino]ethyl}acetamide

The 2-bromo-N-{2-[(3,4-dichlorobenzyl)(methyl)amino]ethyl}acetamide hydrogen bromide salt of point d.) (0.22 g, 0.5 mmol) is treated with the 6-aminobenzthiazol-2-thiol (0.09 g, 0.5 mmol) as described in Example 1.e.) to obtain the title compound which is purified by column chromatography using hexane-ethyl acetate 3:1, then 2:1 mixture as eluent. 0.22 g title compound is obtained in the form of an oil. LC/MS[MH⁺]455 (C₁₉H₂₀Cl₂N₄OS₂ 455.43).

### Example 87

### 2-(6-Aminobenzothiazol-2-ylsulfanyl)-N-{2-[(3,4-dichlorobenzyl)(methyl)amino]-ethyl}propionamide

In the general formula (I) Ar¹ stands for 3,4-dichlorophenyl group, X for methylene group, R¹ for methyl group, Y for ethylene group, R² for hydrogen atom, B for sulphur atom, Z for ethylene group and Ar² for 6-aminobenzothiazol-2-yl group.

### d.) 2-Bromo-N-{2-[(3,4-dichlorobenzyl)(methyl)amino]ethyl}propionamide hydrogen chloride salt

The N-(3,4-dichlorobenzyl)]-N-(methyl)ethane-1,2-diamine (0.23 g, 1 mmol) of Example 86.c.) is treated with 0.19 g (1 mmol) bromopropionyl chloride as described in Example 1.d.) to obtain 0.4 g of the title compound. LC/MS[MH⁺]=367 (C₁₃H₁₇BrCl₂N₂O 368.10).

### e.) 2-(6-Aminobenzothiazol-2-ylsulfanyl)-N-{2-[(3,4-dichlorobenzyl)(methyl)amino]ethyl}propionamide

The 2-bromo-N-{2-[(3,4-dichlorobenzyl)(methyl)amino]ethyl}propionamide hydrogen chloride salt of point d.) (0.39 g, 0.96 mmol) is treated with 6-aminobenzthiazol-2-thiol (0.17 g, 0.96 mmol) as described in Example 1.e.) to obtain the title compound which is purified by column chromatography using chloroform-methanol 15:1 mixture as eluent. 0.16 g title compound is obtained in the form crystals. Mp: 97-100° C.

### Example 88

### N-{3-[(3,4-Dichlorobenzyl)(methyl)amino]-1-methylpropyl}-2-(thiazolo[5,4-b]pyridin-2-yl-sulfanyl)acetamide (1)

In the general formula (I) Ar¹ stands for 3,4-dichlorophenyl group, X and Z for methylene group, R¹ for methyl group, Y for —CH₂—CH₂—CH(CH₃)— group, R² for hydrogen atom, B for sulphur atom, Ar² for thiazolo[5,4-b]pyridin-2-yl group.

### c.) N-(3,4-Dichlorobenzyl)]-N-(methyl)butane-1,3-diamine

### c/1.) 4-[(3,4-Dichlorobenzyl)(methyl)amino]butan-2-one (XI)

The N-(3,4-dichlorobenzyl)methylamine hydrogen chloride salt (4.2 g, 19 mmol) prepared according to Example 1.a.) is dissolved in 10 ml iso-propanol, 1.8 g (60 mmol) paraformaldehyde and 20 ml (340 mmol) acetone are added to it and the reaction mixture is refluxed for 10 hours. After cooling, 15 ml water is added and the pH is set to 10 with 40% sodium hydroxide solution. The aqueous solution is extracted with 3×20 ml ether, the organic layer is dried over sodium sulfate, the solvent is removed and the residue is purified by column chromatography using chloroform-methanol 10:0.5 mixture as eluent. 3.1 g title compound is obtained in the form of an oil. LC/MS[MH⁺]=260 (C₁₂H₁₅Cl₂NO 260.17).

### c/2.) 4-[(3,4-Dichlorobenzyl)(methyl)amino]butan-2-one oxime (XII)

The 4-[(3,4-dichlorobenzyl)(methyl)amino]butan-2-one (2.6 g, 10 mmol) prepared according to point c/1.) is dissolved in 25 ml iso-propanol and the solution of 0.7 g (10 mmol) hydroxylamine hydrochloride in 2.5 ml water is added to it. The reaction mixture is stirred at room temperature for 2 hours. The i-propanol is distilled off, the aqueous residue is alkalinized to pH 10 with 40% sodium hydroxide solution and extracted with 3×20 ml ether. The united organic phase is dried over sodium sulfate, evaporated in vacuum to obtain 2.7 g title compound in the form of an oil. LC/MS[MH⁺]=275 (C₁₂H₁₆N₂Cl₂O 275.18).

### c.) [1-N-(3,4-Dichlorobenzyl)]-N-methylbutan-1,3-diamine

1 g (3.6 mmol) 4-[(3,4-Dichlorobenzyl)(methyl)amino]butan-2-one oxime prepared according to point c/2.) point is hydrogenated in 30 ml ammonia ethanol in the presence of 0.5 g Raney-nickel catalyst. The solvent is removed. 0.79 g title compound is obtained in the form of an oil. LC/MS[MH⁺]=261 (C₁₂H₁₈N₂Cl₂ 261.194).

### d.) 2-Bromo-N-{3-[(3,4-dichlorobenzyl)(methyl)amino]-1-methylpropyl}acetamide hydrogen bromide salt

[1-N-(3,4-Dichlorobenzyl)]-N-methylbutan-1,3-diamine (0.3 g 1.15 mmol) prepared in point c.) is reacted with 0.25 g (1.26 mmol) bromoacetyl bromide according to the procedure as described in Example 1.d.) to obtain 0.26 g title compound. LC/MS[MH⁺] 381 (C₁₄H₁₉BrCl₂N₂O*HBr 463.04)

### e.) N-{3-[(3,4-Dichlorobenzyl)(methyl)amino]-1-methylpropyl}-2-(thiazolo[5,4-b]pyridin-2-ylsulfanyl)acetamide

The 2-Bromo-N-{3-[(3,4-dichlorobenzyl)(methyl)amino]-1-methylpropyl}acetamide hydrogen bromide salt (0.46 g, 1 mmol) of point d.) is reacted with 0.16 g (1 mmol) thiazolo[5,4-b]pyridin-2-thiol according to the procedure as described in Example 1.e.) to obtain 0.17 g title compound in the form of an oil. LC/MS[MH⁺] 469 (C₂₀H₂₂Cl₂N₄OS₂ 469.46).

### Examples 89-91

The compounds of Table 4. are prepared according to the procedure as described in Example 88.

TABLE 4

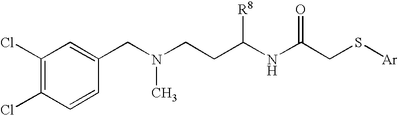

Ar¹ = 3,4-dichlorophenyl
X = —CH₂—
R¹ = —CH₃
Y = —CH₂—CH₂—CH(R⁸)—

[symbol]

Z = —CH₂—

| | | | Mp | |
|---|---|---|---|---|
| Example | R⁸ | Ar² | (° C.) | [MH⁺] |
| 89. | Me(rac) | 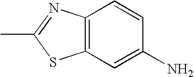 | 85-87 | |
| 90. | Me(enant.) | 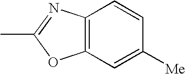 | 200 | |
| 91. | Me(enant.) | 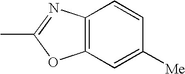 | 199 | |

### Example 92

### N-{3-[(3,4-Dichlorobenzyl)(methyl)amino]-1-methylpropyl}-2-(4-methylbenzoxazol-2-ylsulfanyl)acetamide

In the general formula (I) Ar¹ stands for 3,4-dichlorophenyl group, X and Z for methylene group, R¹ for methyl group, Y for —CH₂—CH₂—CH(CH₃)— group, R² for hydrogen atom, B for sulfur atom, Ar² for 4-methylbenzoxazol-2-yl-group.

0.44 g (2 mmol) (4-methylbenzoxazol-2-ylsulfonyl)acetic acid is dissolved in 6 ml chloroform and 0.2 g (2 mmol) N-methylmorpholine is added to it. The mixture is cooled to −10° C., 0.27 g (2 mmol) tert-butyl chloroformate and after 15 minutes of stirring 0.55 g (2.11 mM) N-(3,4-dichlorobenzyl)]-N-(methyl)butan-1,3-diamine in 3 ml chloroform are added. The reaction mixture is stirred for 30 minutes under cooling and 30 minutes at room temperature. The solution is then washed with water and with 5% potassium hydrogen sulfate solution, dried over sodium sulfate and evaporated in vacuum. The resulting oil is dissolved in ethyl acetate and transformed into the oxalate salt. In the form of white crystals 700 mg title compound is obtained.

Mp.: 108-111° C.

### Example 93

### N-{3-[(3,4-Dichlorobenzyl)(methyl)amino]-1-methylpropyl}-2-(6-methylbenzoxazol-2-ylsulfanyl)acetamide oxalate salt

In the general formula (I) Ar¹ stands for 3,4-dichlorophenyl group, X and Z for methylene group, R¹ for methyl group, Y for —CH₂—CH₂—CH(CH₃)— group, R² for hydrogen atom, B for sulphur atom, Ar² for 6-methylbenzoxazol-2-yl group.

According to the procedure described in Example 92., starting from 0.4 g (1.83 mmol) (6-methylbenzoxazol-2-ylsulfonyl)acetic acid, 367 mg title compound is obtained as white crystals. Mp: 148-150° C.

### Example 94

### 2-(Benzothiazol-2-ylsulfanyl)-N-{3-[(3,4-dichlorobenzyl)(methyl)amino]propyl}-N-methylacetamide (I)

In the general formula (I) Ar¹ stands for 3,4-dichlorophenyl group, X and Z for methylene group, R¹ for methyl group, Y for 1,3-propylene group, R² for methyl group, B for sulphur atom, Ar² for benzothiazol-2-yl group.

### c.) N-(3,4-Dichlorobenzyl)-N,N′-(dimethyl)propan-1,3-diamine

1.5 ml (12 mmol) N,N′-(dimethyl)propylamine is dissolved in 15 ml acetonitrile and 2.5 ml (18 mmol) triethylamine, then dropwise 1.4 ml (10 mmol) 3,4-chlorobenzyl chloride is added to it. The reaction mixture is heated under reflux conditions for 2 hours. The solution is evaporated, the residue is dissolved in dichloromethane, the insoluble salts are filtered off, the organic phase is washed with water, dried over sodium sulfate, evaporated in vacuum and purified by column chromatography. Thus, 0.8 g title compound is obtained in the form of an oil. LC/MS[MH⁺] 261 C₁₂H₁₈Cl₂N₂ 261.20)

### d.) 2-Bromo-N-{3-[(3,4-dichlorobenzyl)(methyl)amino]propyl}-N-methyl acetamide hydrogen bromide salt

The N-(3,4-Dichlorobenzyl)-N,N′-(dimethyl)propan-1,3-diamine of point c.) (0.8 g 3 mmol) is reacted with 0.3 ml 3.4 mmol bromoacetyl bromide, according to the procedure as described in Example 1.d.) to obtain 0.46 g title compound as white crystals. Mp.: 142-146° C.

### e.) 2-(Benzothiazol-2-ylsulfanyl)-N-{3-[(3,4-dichlorobenzyl)(methyl)amino propyl}-N-methylacetamide

The 2-Bromo-N-{3-[(3,4-dichlorobenzyl)(methyl)amino]propyl}-N-methyl acetamide hydrogen bromide salt (0.083 g 0.5 mmol) of point d.) is reacted with 0.23 g (0.5 mmol) benzothiazol-2-thiol according to the procedure as described in Example 1.e.) to obtain 0.17 g title compound in the form of an oil. LC/MS[MH⁺]=468 (C₂₁H₂₃Cl₂N₃OS₂ 468.47).

### Example 95

### 2-(6-Aminobenzothiazol-2-ylsulfanyl)-N-{3-[[3-(3,4-dichlorophenyl)propyl](methyl)amino]propyl}acetamide (I)

In the general formula (I) Ar¹ stands for 3,4-dichlorophenyl group, X and Y for 1,3-propylene group, Z for methylene group, R¹ for methyl group, R² for hydrogen atom, B for sulphur atom, Ar² for 6-aminobenzothiazol-2-yl group.

### a.) [3-(3,4-Dichlorophenyl)propyl]methylamine

### a/1.) 3-(3,4-Dichlorophenyl)propionaldehyde

To the solution of 10 ml pyridine and 100 ml dichloromethane under ice-cooling 6.3 g (63 mmol) chrom trioxide is added and the mixture is stirred at room temperature for 1 hour. To this mixture is added the solution of 1.4 g (7 mmol) 3-(3,4-dichlorophenyl)propan-1-ol in 22 ml dichloromethane and stirring is continued for 15 minutes. The solid material is filtered off, washed with 3×35 ml ether. The ether mother liquor is washed with 3×35 ml 5% sodium hydroxide solution, with 3×35 ml 2N hydrochloric acid solution and finally with 3×35 ml saturated sodium hydrogencarbonate solution, dried over sodium sulfate and evaporated to obtain 1 g title compound in the form of an oil. LC/MS[MH⁺]=203 (C₉H₈Cl₂O 203.07).

### a.) [3-(3,4-Dichlorophenyl)propyl]methylamine

The 3-(3,4-Dichlorophenyl)propionaldehyde of point a/1.) (1 g, 5 mmol) is treated according to the procedure as described in Example 1.a.) with the exception that the hydrogen chloride salt is not formed. Thus, 0.8 g title compound is obtained.

LC/MS[MH⁺]=218 (C₁₀H₁₃Cl₂N 218.12).

### b.) 3-{[3-(3,4-Dichlorophenyl)propyl](methyl)amino}propionitrile

The [3-(3,4-Dichlorophenyl)propyl]methylamine (0.85 g, 3.9 mmol) of point a.) is reacted with 0.2 g (3.9 mmol) acryl nitrile according to the procedure as described in Example 1.b.). Thus, 0.77 g title compound is obtained in the form of an oil. LC/MS[MH⁺]=271 (C₁₃H₁₆Cl₂N₂ 271.19).

### c.) N-[3-(3,4-Dichlorophenyl)propyl]-N-(methyl)propan-1,3-diamine

The 3-{[3-(3,4-Dichlorophenyl)propyl](methyl)amino}propionitrile (0.77 g, 2.84 mmol) of point b.) is treated as described in Example 1.c.) to obtain 0.7 g title compound in the form of an oil. LC/MS[MH⁺]=275 (C₁₃H₂₀Cl₂N₂ 275.22).

### d.) 2-Bromo-N-{3-[[3-(3,4-dichorophenyl)propyl](methyl)amino]propyl}acetamide hydrogen bromide salt

The N-[3-(3,4-Dichlorophenyl)propyl]-1′-N-(methyl)propan-1,3-diamine (0.27 g, 1 mmol) of point c.) is reacted with 0.22 g (1.1 mmol) bromoacetyl bromide according to the procedure described in Example 1.d.) to obtain 0.49 g title compound which cannot be crystallized. LC/MS[MH⁺]=395 (C₁₅H₂₁BrCl₂N₂O*HBr 477.06)

### e.) 2-(6-Aminobenzothiazol-2-ylsulfanyl)-N-{3-[[3-(3,4-dichlorophenyl)propyl](methyl)amino]propyl}acetamide

The 2-Bromo-N-{3-[[3-(3,4-dichorophenyl)propyl](methyl)amino]propyl}acetamide hydrogen bromide salt (0.31 g 0.65 mmol) of point d.) is reacted with 0.12 g (0.65 mmol) 6-amino-benzothiazol-2-thiol according to the procedure described in Example 1.e). After purification by column chromatography 0.05 g title compound is obtained in the form of an oil. LC/MS[MH⁺]=497 (C₂₂H₂₆Cl₂N₄OS₂ 497.51)

### Example 96

### 2-(6-Aminobenzothiazol-2-ylsulfanyl)-N-{3-[[3-(3,4-dichlorophenyl)-1′-methylpropyl]-(methyl)amino]propyl}acetamide (I)

In the general formula (I) Ar¹ stands for 3,4-dichlorophenyl group, X for —CH₂—CH₂—CH(CH₃)— group, Y for propylene group, Z for methylene group, R¹ for methyl group, R² for hydrogen atom, B for sulfur atom, Ar² for 6-aminobenzothiazol-2-yl group.

### a.) [3-(3,4-Dichlorophenyl)-1-methylpropyl]methylamine

### a/1.) 4-(3,4-Dichlorophenyl)butan-2-one

(Rosowsky A. et al.: J. Med. Chem. 1973, 16, 191-194)

9.7 g (50 mmol) 3,4-dichlorobenzyl chloride and 5.5 g (55 mmol) pentane-2,4-dione is dissolved in 50 ml methanol and the solution is heated under reflux for 24 hours. After cooling, methanol is removed in vacuum, the residue is extracted with 50 ml water and 3×15 ml ether. The organic phase is dried over sodium sulfate and evaporated in vacuum. The residue is distilled under 5 Hgmm at 120° C. 5.9 g title compound is obtained in the form of an oil. LC/MS[MH⁺]=217 (C₁₀H₁₀Cl₂O 217.94).

### a.) [3-(3,4-Dichlorophenyl)-1-methylpropyl]methylamine

The 4-(3,4-Dichlorophenyl)butan-2-one (4.3 g, 20 mmol) of point a/1.) is treated according to the procedure described in Example 1.a.) to obtain 4.2 g title compound in the form of an oil. LC/MS[MH⁺]=232 (C₁₁H₁₅Cl₂N 232.15).

### b.) 3-{[3-(3,4-Dichlorophenyl)-1-methylpropyl](methyl)amino}propionitrile

The [3-(3,4-Dichlorophenyl)-1-methylpropyl]methylamine (4.18 g, 18 mmol) of point a.) is reacted with 0.96 g (18 mmol) acryl nitrile according to the procedure described in Example 1. b.) to obtain 4 g title compound in the form of an oil. LC/MS[MH⁺]=285 (C₁₄H₁₈Cl₂N₂ 285.21).

### c.) N-[3-(3,4-Dichlorophenyl)-1-methylpropyl]-N-methylpropan-1,3-diamine

The 3-{[3-(3,4-Dichlorophenyl)-1-methylpropyl](methyl)amino}propionitrile (3.15 g, 11 mmol) of point b.) is treated as described in Example 1.c.) to obtain 0.62 g title compound in the form of an oil. LC/MS[MH⁺]=289 (C₁₄H₂₂Cl₂N₂ 289.25).

### d.) 2-Bromo-N-(3-{[3-(3,4-dichlorophenyl)-1-methylpropyl](methyl)amino}propyl)acetamide hydrogen bromide salt

The N-[3-(3,4-Dichlorophenyl)-1-methylpropyl]-N-methylpropan-1,3-diamine (0.57 g, 2 mmol), of point c.) is reacted with 0.44 g (2.2 mol) bromoacetyl bromide according to the procedure described in Example 1. d.) to obtain 1 g title compound. LC/MS[MH⁺]=408 (C₁₇H₂₄BrCl₂NO*HBr 491.09).

### e.) 2-(6-Aminobenzthiazol-2-ylsulfanyl)-N-(3-{[3-(3,4-dichlorophenyl)-1-methylpropyl](methyl)amino}propyl)acetamide

The 2-Bromo-N-(3-{[3-(3,4-dichlorophenyl)-1-methylpropyl](methyl)amino}propyl)acetamide hydrogen bromide (0.2 g 0.5 mmol) of point d.) is reacted with 0.09 g (0.5 mmol) 6-aminobenzothiazol-2-thiol according to the procedure described in Example 1.e.). After purification by column chromatography, 0.09 g title compound is obtained in the form of an oil. LC/MS[MH⁺]=511 (C₂₃H₂₈Cl₂N₄OS₂ 511.54).

### Example 97

### N-{3-[(3,4-dichlorobenzyl)(methyl)amino]propyl}-2-(4-methylbenzoxazol-2-yl)sulfinyl]acetamide

In the general formula (I) Ar¹ stands for 3,4-dichlorophenyl group, X and Z for methylene group, Y for 1,3-propylene group, R¹ for methyl group, R² for hydrogen atom, B for SO— group, Ar² for 4-methylbenzoxazol-2-yl group.

To the solution of 0.1 g (0.18 mmol) N-{3-[(3,4-dichlorobenzyl)(methyl)amino]propyl}-2-(4-methylbenzoxazol-2-yl)sulfanyl]acetamide in 2 ml dichloromethane under ice-water cooling 0.045 g (0.2 mmol) meta-chloroperbenzoic acid is added. The reaction mixture is stirred for 1 hour, then neutralized with solid potassium carbonate. The precipitated salts are filtered off, the dichloromethane solution is evaporated. The residue is crystallized with ether, filtered off, purified by column chromatography using chloroform-methanol 9:1 mixture as eluent. Thus, 60 mg title compound is obtained in the form of crystals. Mp.: 155-156° C.

### Example 98

### N-{3-[(3,4-dichlorobenzyl)(methyl)amino]propyl}-2-(4-methylbenzoxazol-2-yl)sulfonyl]acetamide

In the general formula (I) Ar¹ stands for 3,4-dichlorophenyl group, X and Z for methylene group, Y for propylene group, R¹ for methyl group, R² for hydrogen atom, B for SO₂ group, Ar² for 4-methylbenzoxazol-2-yl group.

To the solution of 0.1 g (0.18 mmol) N-{3-[(3,4-dichlorobenzyl)(methyl)amino]propyl}-2-(4-methylbenzoxazol-2-yl)sulfanyl]acetamide in 2 ml dichloromethane, under ice-water cooling 0.09 g (0.4 mmol) meta-chloroperbenzoic acid is added. The reaction mixture is stirred for 1 hour, then neutralized with solid potassium carbonate. The precipitated salts are filtered off, the dichloromethane solution is evaporated. The residue is crystallized with ether to obtain the title compound in the form of crystals.

LC/MS[MH⁺]=484 (C₂₁H₂₃Cl₂N₃O₄S 484.41).

### Example 99

In known methods the tablet of the following composition is prepared:

| | |
|---|---|
| Active component: | 40 mg |
| Lactose: | 35 mg |
| Avicel: | 21 mg |
| Crospovidone: | 3 mg |
| Magnesium stearate: | 1 mg |

### Example 100

### A.) Human Recombinant CCR3 Receptor (hr-CCR3) Binding Assay

The CCR3 receptor antagonist effect of the compounds of general formula (I) was examined on eotaxin binding test on hCCR3 receptor expressing recombinant K562 and RBL2H3 cells. To the tests Eotaxin labelled with radioactive iodine ¹²⁵I-(2200 Ci/mmol) was used.

In the assay 200000 cells are incubated in the presence of 0.11 nM ¹²⁵I-Eotaxin, incubation: 60 minutes at 37° C. Composition of the assay buffer: RPMI-1640 medium, pH=7.6 (GIBCO), [containing 80 mg CHAPS, 500 BSA (protease free), 100 mg Gelatine, 3 ml 25 mM HEPES in 100 ml RPMI]. The test compounds are dissolved in DMSO, the stock solution is diluted with the assay buffer. The final DMSO concentration is not more than 1%. The assays are performed in deep-well plates. The cells are incubated with the test compounds for 15 minutes, then the labelled eotaxin is added. The non-specific binding is determined in the presence of 200 nM non-labelled eotaxin. After 1 hour of incubation, 500 μl ice-cold assay buffer containing 0.5 M NaCl solution is added. The reaction is terminated by centrifugation in plate centrifuge (JUAN) at 3600 g for 6 minutes. The supernatants are poured off by tuning the plates in upside-down position. The remaining droplets were blotted with tissue paper. For solubilization 200 μl 0.5 M NaOH solution is added to the pellets. After 1 hour of solubilization at room temperature the radioactivity of 150 μl solubilized solution is counted in gamma counter (1470 Wizard, Wallac).

The radioactivity of the solution is in direct ratio with the number of the receptors of the cells, with the amount of the bound ¹²⁵I-Eotaxin and with the activity of the tested antagonist.

The specific binding is calculated as the difference between the total and the non-specific bindings. The activity of the compounds is calculated from the specific binding and from the binding measured in the presence of the antagonist molecule.

The activity of the compounds is characterized with the IC₅₀ value.

### B.) Investigation of Ca²⁺ Mobilization in hCCR3-RBL and hCCR3K562 Cells

HCCR3-K562 and hCCE3-RBL2H3 cells in 40000 cells/well density (number of cells in one well of the microplate) are cultured for 24 hours. The cells are washed and loaded with calcium indicator dye (Calcium Plus assay Kit, Molecular Devices). The cells are incubated in the presence of the dye for 60 minutes while loading takes place. The dye is a fluorescent calcium indicator, which sensitively indicates the intracellular calcium concentration. The intracellular calcium concentration is in direct ratio with the fluorescent signal of the sample. The experiments are performed in a BMG NOVOSTAR apparatus, at excitation and emission wavelengths.

The selective agonists used in the experiments are:

Eotaxin

Eotaxin-2

Eotaxin-3

RANTES

Following the addition of the selective agonist, the intracellular calcium concentration in the cells significantly increases which can be monitored with the help of the fluorescent signal. In the experiments an agonist concentration is used which causes a 75% calcium signal compared to the maximum attainable signal.

Antagonists are added 15 minutes before the agonist treatment.

The change of the fluorescent signal is monitored for 30 seconds, during that period the process takes place.

The intensity of the maximum signal following the addition of the agonist is compared with the calcium signal obtained after the addition of the same agonist, but in the presence of the inhibitor.

The activity of the compounds is characterized with the IC₅₀ values.

On the basis of tests A and B the compounds of general formula (I) were found biologically active. IC₅₀ values of the most potent compounds are in the range of 0.5 nM to 500 nM. Of these compounds, the especially favoured molecules have IC₅₀ values between 0.5 nM and 15 nM.

1. A compound of the general formula (I), 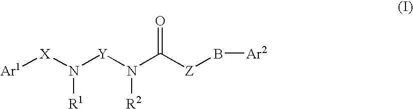
wherein
B stands for sulfur atom, or —SO— or —SO₂— group;
Ar¹ represents phenyl- or naphthyl group, optionally substituted with one or more identical or non-identical substituents selected from the group consisting of straight or branched C₁₋₄ alkyl group, halogen atom, straight or branched C₁₋₄ alkoxy group, trifluoromethyl group, cyano group, nitro group, hydroxyl group, C₁₋₂ alkylenedioxy group, amino group, and amino group—substituted with one or two identical or non-identical straight or branched C₁₋₄ alkyl group—;
X and Y independently mean a straight C₁₋₄ alkylene group optionally substituted with one or more identical or non-identical straight or branched C₁₋₄ alkyl group;
Z stands for a straight C₁₋₄ allylene group optionally substituted with one or more identical or non-identical straight or branched C₁₋₄ alkyl group or phenyl group;
R¹ and R² independently mean hydrogen atom or a straight or branched C₁₋₄ alkyl group; and
Ar² stands for phenyl group, benzyl group, thienyl group or furyl group, each optionally substituted with one or more identical or non-identical substituents selected from the group consisting of straight or branched C₁₋₄ alkyl group, straight or branched C₁₋₄ alkoxy group, hydroxyl group, amino group, amino group—substituted with one or two identical or non-identical straight or branched C₁₋₄ alkyl or aralkyl group—, trifluoromethyl group, cyano group, C₁₋₂ alkylenedioxy group, and halogen atom;
5- or 6-membered heterocyclic ring group containing one, two, three or four nitrogen atoms, or two nitrogen atoms and one oxygen atom, or one nitrogen atom and one oxygen atom, or one nitrogen atom and one sulphur atom, optionally substituted with one or more identical or non-identical substituents selected from the group consisting of straight or branched C₁₋₄ alkyl group, C₃₋₆ cycloalkyl group, 1,4-butylene group, straight or branched C₁₋₄ alkoxy group, halogen atom, nitro group, cyano group, hydroxyl group, amino group, amino group—substituted with one or two identical or non-identical straight or branched C₁₋₄ alkyl- or aralkyl group—, trifluoromethyl group, C₁₋₄ hydroxyalkyl group, phenyl group—optionally substituted with one or more straight or branched C₁₋₄ alkyl group, halogen atom or benzyloxy group—, benzyl group—optionally substituted with one or more straight or branched C₁₋₄ alkyl group, straight or branched C₁₋₄ alkoxy group or halogen atom—, furyl group, thienyl group, pyridyl group, —CO—O—R³ group—where R³ stands for straight or branched C₁₋₄ alkyl group—, —NH—CH₂—CO—O—R⁴ group—where R⁴ stands for straight or branched C₁₋₄ alkyl group—, —C₆H₄—NH—CO—R⁵ group—where R⁵ stands for straight or branched C₁₋₄ alkyl group—, and oxo group,
benzologue of the 5- or 6-membered heterocyclic ring group where the benzene ring may optionally be further substituted with one or more identical or non-identical substituents selected from the group consisting of straight or branched C₁₋₄ alkyl group, straight or branched C₁₋₄ alkoxy group, hydroxyl group, trifluoromethyl group, cyano group, nitro group, C₁₋₂ alkylenedioxy group, amino group, amino group—substituted with one or two identical or non-identical straight or branched C₁₋₄ alkyl or aralkyl group—, halogen atom, sulfonyl group, and sulfonamide group, or
5- or 6-membered heterocyclic ring group containing one, two or three nitrogen atoms, or one nitrogen atom and one oxygen atom, or one nitrogen atom and one sulphur atom, condensed with a 6-membered heteroaromatic ring group containing one or two nitrogen atoms, optionally substituted with one or more identical or non-identical substituents selected from the group consisting of straight or branched C₁₋₄ alkyl group, straight or branched C₁₋₄ alkoxy group, halogen atom, nitro group, cyano group, hydroxyl group, amino group, amino group—substituted with one or two, identical or non-identical straight or branched C₁₋₄ alkyl group or benzyl group—, and 1-(C₁₋₄-alkylcarbonyl)-2-phenylethyl group;
with the proviso that if B stands for —SO₂— group and Z means a straight C₁₋₄ alkylene group optionally substituted with one or more identical or non-identical straight or branched C₁₋₄ alkyl group—; and with the further proviso that when Ar¹ represents phenyl group, X means methylene group substituted with one methyl group, Y means propylene group, Z stands for propylene or butylene group, R¹ means methyl group, R² means hydrogen atom and Ar² stands for phenyl group, B is different from —SO₂— group or
a salt, solvate or isomer thereof, salt of the solvate or isomer thereof, or solvate of the isomer thereof.

2. The compound of the general formula (I) according to claim 1, wherein
Ar¹ stands for phenyl group, optionally substituted with one or more halogen atom; and
Ar² stands for phenyl group;
5- or 6-membered heterocyclic ring group containing one, two, three or four nitrogen atoms, or two nitrogen atoms and one oxygen atom, or one nitrogen atom and one oxygen atom, or one nitrogen atom and one sulphur atom, optionally substituted with one or more identical or non-identical substituents selected from the group consisting of straight or branched C₁₋₄ alkyl group, C₃₋₆ cycloalkyl group, 1,4-butylene group, cyano group, amino group, trifluormethyl group, C₁₋₄ hydroxyalkyl group, phenyl group—optionally substituted with one or more straight or branched C₁₋₄ alkyl group, halogen atom or benzyloxy group—, benzyl group—optionally substituted with straight or branched C₁₋₄ alkoxy group or halogen atom—, thienyl group, furyl group, pyridyl group, —CO—O—R³ group—where R³ stands for straight or branched C₁₄ allyl group—, —NH—CH₂—CO—O—R⁴ group—where R⁴ stands for straight or branched C₁₋₄ alkyl group—, —C₆H₄—NH—CO—R⁵ group—where R⁵ stands for straight or branched C₁₋₄ alkyl group—, and oxo group;
benzologue of the 5- or 6-membered heterocyclic ring group where the benzene ring may optionally be further substituted with one or more identical or non-identical substituents selected from the group consisting of straight or branched C₁₋₄ alkyl group, straight or branched C₁₋₄ alkoxy group, trifluoromethyl group, nitro group, C₁₋₂ alkylenedioxy group, amino group, amino group—substituted with one or two identical or non-identical straight or branched C₁₋₄ alkyl group—, halogen atom, and sulfonyl group; or
5-membered heterocyclic ring containing two or three nitrogen atoms, or one nitrogen atom and one oxygen atom, or one nitrogen atom and one sulphur atom condensed with a 6-membered heteroaromatic ring group containing one or two nitrogen atoms, optionally substituted with one or more identical or non-identical substituents selected from the group consisting of straight or branched C₁₋₄ alkyl group, straight or branched C₁₋₄ alkoxy group, amino group—substituted with one or two, identical or non-identical straight or branched C₁₋₄ alkyl group or benzyl group—, and 1-(C₁₋₄-alkylcarbonyl)-2-phenylethyl group;
with the proviso that if B stands for SO₂ group, Z means a straight C₁₋₄ alkylene group—optionally substituted with one or more identical or non-identical straight or branched C₁₋₄ alkyl group, and with the further proviso that when Ar¹ represents phenyl group, X means methylene group substituted with one methyl group, Y means propylene group, Z stands for propylene or butylene group, R¹ means methyl group, R² means hydrogen atom and Ar² stands for phenyl group, B is different from —SO₂— group; or
a salt, solvate or isomer thereof, salt of the solvate or isomer thereof, or solvate of the isomer thereof.

3. The compound of the general formula (I) according to claim 1, wherein
B stands for sulfur atom, or —SO— group;
Ar¹ stands for phenyl group, optionally substituted with one or more halogen atom; and
Ar² stands for
5- or 6-membered heterocyclic ring group containing one, two or three nitrogen atoms, or two nitrogen atoms and one oxygen atom, or one nitrogen atom and one oxygen atom, or one nitrogen atom and one sulphur atom, optionally substituted with one or more identical or non-identical substituents selected from the group consisting of straight or branched C₁₋₄ alkyl group, C₃₋₆ cycloalkyl group, 1,4-butylene group, cyano group, amino group, trifluormethyl group, phenyl group—optionally substituted with one or more straight or branched C₁₋₄ alkyl group—, thienyl group, furyl group, pyridyl group, and —CO—O—R³ group—where R³ stands for straight or branched C₁₋₄ alkyl group—;
benzologue of the 5- or 6-membered heterocyclic ring group where the benzene ring may optionally be further substituted with one or more identical or non-identical substituents selected from the group consisting of straight or branched C₁₋₄ alkyl group, straight or branched C₁₋₄ alkoxy group, C₁₋₂ alkylenedioxy group, amino group, amino group—substituted with one or two identical or non-identical straight or branched C₁₋₄ alkyl group—, and halogen atom; or
5-membered heterocyclic ring containing two or three nitrogen atoms, or one nitrogen atom and one oxygen atom, or one nitrogen atom and one sulphur atom condensed with a 6-membered heteroaromatic ring group containing one or two nitrogen atoms, optionally substituted with one or more identical or non-identical substituents selected from the group consisting of straight or branched C₁₋₄ alkyl group, straight or branched C₁₋₄ alkoxy group, amino group—substituted with one or two, identical or non-identical straight or branched C₁₋₄ alkyl group or benzyl group—; or
a salt, solvate or isomer thereof, salt of the solvate or isomer thereof, or solvate of the isomer thereof.

4. The compound of the general formula (I) according to claim 1 selected from:
N-{3-[(3,4-Dichlorobenzyl)(methyl)amino]propyl}-2-(5-dimethylaminothiazolo[5,4-b]pyridin-2-ylsulfanyl)acetamide;
N-{3-[(3,4-Dichlorobenzyl)(methyl)amino]propyl}-2-(5-dimethylaminothiazolo[5,4-d]pyrimidin-2-ylsulfanyl)acetamide;
2-(6-Aminobenzoxazol-2-ylsulfanyl)-N-{3-[(3,4-dichlorobenzyl)(methyl)amino]propyl}acetamide;
N-{3-[(3,4-Dichlorobenzyl)(methyl)amino]propyl}-2-(6-methoxybezoxazol-2-ylsulfanyl)acetamide;
N-{3-[(3,4-Dichlorobenzyl)(methyl)amino]propyl}-2-(1,6-dimethyl-1H-benzimidazol-2-ylsulfanyl)acetamide;
N-{3-[(3,4-Dichlorobenzyl)(methyl)amino]propyl}-2-(oxazolo[5,4-b]pyridin-2-ylsulfanyl)acetamide;
2-(6-Aminobenzthiazol-2-ylsulfanyl)-N-{3-[(3,4-dichlorobenzyl)(methyl)amino]propyl}acetamide;
2-(Benzthiazol-2-ylsulfanyl)-N-{3-[(3,4-dichlorobenzyl)(methyl)amino]propyl}acetamide;
N-{3-[(3,4-Dichlorobenzyl)(methyl)amino]propyl}-2-(6-methylbezoxazol-2-ylsulfanyl)acetamide;
N-{3-[(3,4-Dichlorobenzyl)(methyl)amino]propyl}-2-(thiazolo[5,4-b]pyridin-2-ylsulfanyl)acetamide;
2-(Benzoxazol-2-ylsulfanyl)-N-{3-[(3,4-dichlorobenzyl)(methyl)amino]propyl}acetamide;
N-{3-[(3,4 Dichlorobenzyl)(methyl)amino]propyl}-2-(5-methoxybenzothiazol-2-ylsulfanyl)acetamide;
N-{3-[(3,4-Dichlorobenzyl)(methyl)amino]propyl}-2-(6-ethoxybenzothiazol-2-ylsulfanyl)acetamide;
N-{3-[(3,4-Dichlorobenzyl)(methyl)amino]propyl}-2-(5-ethylaminothiazolo[5,4-d]pyrimidin-2-ylsulfanyl)acetamide;
N-{3-[(3,4-Dichlorobenzyl)(methyl)amino]propyl}-2-(5-ethylaminothiazolo[5,4-b]pyridin-2-ylsulfanyl)acetamide;
N-{3-[(3,4-Dichlorobenzyl)(methyl)amino]propyl}-2-(5-isopropylaminothiazolo[5,4-d]pyrimidin-2-ylsulfanyl)acetamide;
N-{3-[(3,4-Dichlorobenzyl)(methyl)amino]propyl}-2-(5-isopropylaminothiazolo[5,4-b]pyridin-2-ylsulfanyl)acetamide;
2-(5-Benzylaminothiazolo[5,4-b]pyridin-2-ylsulfanyl)-N-{3-[(3,4-dichlorobenzyl) (methyl)amino]propyl}acetamide;
2-(6-Aminobenzthiazol-2-ylsulfanyl)-N-{3-[(3,4-dichlorobenzyl)(methyl)amino]butyl}acetamide;
2-(6-Aminobenzthiazol-2-ylsulfanyl)-N-{3-[(3,4-dichlorobenzyl)(methyl)amino]propyl}butyramide;
N-{3-[(3,4-Dichlorobenzyl)(methyl)amino]propyl}-2-(6-methyl-1H-benzimidazol-2-ylsulfanyl)acetamide;
N-{3-[(3,4-Dichlorobenzyl)(methyl)amino]propyl}-2-(quinazolin-2-ylsulfanyl)acetamide;
2-(5-Benzylaminothiazolo[5,4-d]pyrimidin-2-ylsulfanyl)-N-{3-[(3,4-dichlorobenzyl) (methyl)amino]propyl}acetamide;
2-(6-Aminobenzthiazol-2-ylsulfanyl)-N-{2-[(3,4-dichlorobenzyl)(methyl)amino]ethyl}acetamide;
3-(6-Aminobenzthiazol-2-ylsulfanyl)-N-{3-[(3,4-dichlorobenzyl)(methyl)amino]propyl}propionamide;
2-(6-Aminobenzthiazol-2-ylsulfanyl)-N-{3-[(3,4-dichlorobenzyl)(methyl)amino]propyl}acetamide;
2-(6-Aminobenzthiazol-2-ylsulfanyl)-N-{3-[(3,4-dichlorophenyl)propyl](methyl)amino-propyl}acetamide;
2-(6-Aminobenzthiazol-2-ylsulfanyl)-N-{3-[(3,4-dichlorobenzyl)(methyl)amino]butyl}acetamide;
N-{3-[(3,4-Dichlorobenzyl)(methyl)amino]-1-methylpropyl}-2-(6-methylbenzoxazol-2-ylsulfanyl)acetamide;
N-{3-[(3,4-Dichlorobenzyl)(methyl)amino]-1-methylpropyl}-2-(thiazolo[5,4-b]pyridin-2-ylsulfanyl)acetamide;
2-(6-Aminobenzthiazol-2-ylsulfanyl)-N-{3-[(3,4-dichlorobenzyl)(methyl)amino]-2-methylpropyl}acetamide;
2-(6-Aminobenzthiazol-2-ylsulfanyl)-N-{3-[(3,4-dichlorobenzyl)(methyl)amino]-1-methylpropyl}acetamide;
2-(6-Aminobenzthiazol-2-ylsulfanyl)-N-{3-[(3,4-dichlorobenzyl)(methyl)amino]propyl}-N-methylacetamide;
(+)N-{3-[(3,4-Dichlorobenzyl)(methyl)amino]-1-methylpropyl}-2-(6-methylbenzoxazol-2-ylsulfanyl)acetamide;
(−)N-{3-[(3,4-Dichlorobenzyl)(methyl)amino]-1-methylpropyl}-2-(6-methylbenzoxazol-2-ylsulfanyl)acetamide;
2-(6-Aminobenzthiazol-2-ylsulfanyl)-N-{3-[(3,4-dichlorobenzyl)(methyl)amino]propyl}-propionamide; and
N-{3-[(3,4-Dichlorobenzyl)(methyl)amino]propyl}-2-(4-methylbenzoxazol-2-yl)sulfinyl]acetamide; or
a salt, solvate or isomer thereof, salt of the solvate or isomer thereof, or solvate of the isomer thereof.

5. A process for the preparation of the compound of the general formula (I) according to claim 1, or a salt, solvate or isomer thereof comprising
a) reacting a halogen compound of the general formula (III), 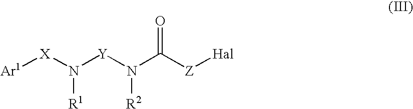
wherein Ar¹, X, Y, Z, R¹, and R² are as defined in claim 1, and Hal stands for halogen atom, with a compound of the general formula (II) wherein B and Ar² are as defined in claim 1,
HB—Ar² (II),
b) reacting an amine of the general formula (VIII) wherein Ar¹, X, and R¹ are as defined in claim 1, 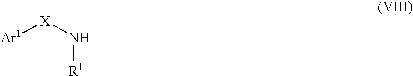
with a halogen compound of the general formula (XVI) wherein Y, Z, R², B and Ar² are as defined in claim 1, and Hal stands for halogen atom 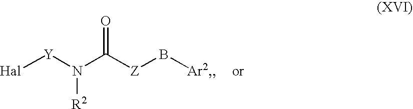
or
c) reacting a diamino compound of the general formula (V) wherein Ar¹, X, Y, R², and R² are as defined in claim 1, 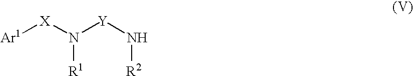
with a carboxylic acid derivative of the general formula (XVII) wherein Z, B, and Ar² are as defined in claim 1, and W represents halogen atom, hydroxyl group, —OR¹¹ group—where R¹¹ stands for C₁₋₄-alkyl group—or —O—CO-Z-B—Ar² group 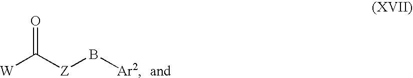
and
optionally transforming a substituent of the compound of the general formula (I) thus obtained according to step a), b) or c) into another by using a known method and/or the resultant compound of the general formula (I) obtained according to step a, b or c is transformed into a salt or solvate thereof, or liberated from a salt or solvate thereof and/or resolved into an optically active isomer, or the optically active isomer is transformed into the racemic compound and if desired separating structural isomers from each other.

6. The process according to step a) or b) of claim 5, wherein the reacting is carried out in the presence of a base.

7. The process according to step c) of claim 5, wherein in the compound of the general formula (XVII) W is chloride atom and the reacting is carried out in the presence of a base.

8. The process according to step c) of claim 5, wherein in the compound of the general formula (XVII) W is hydroxyl group, and the reacting is carried out in the presence of an activating agent.

9. The process according to claim 8, wherein the activating agent is dicyclohexylcarbodiimide, pivalyl chloride, ethyl chloroformate, isobutyl chloroformate, carbonyldiimidazole, or benzotriazol-1-yl-oxy-tripyrrolidinophosphonium hexafluorophosphate.

10. A pharmaceutical preparation wherein it contains one or more of the compounds of the general formula (I), according to claim 1, or a salt, solvate or isomer thereof, salt of the solvate or isomer thereof, or solvate of the isomer thereof, and one or more excipients used in the pharmaceutical industry.

11. The pharmaceutical preparation according to claim 10, wherein the one or more compounds of the general formula (I) is/are selected from
N-{3-[(3,4-Dichlorobenzyl)(methyl)amino]propyl}-2-(5-dimethylaminothiazolo[5,4-b]pyridin-2-ylsulfanyl)acetamide;
N-{3-[(3,4-Dichlorobenzyl)(methyl)amino]propyl}-2-(5-dimethylaminothiazolo[5,4-d]pyrimidin-2-ylsulfanyl)acetamide;
2-(6-Aminobenzoxazol-2-ylsulfanyl)-N-{3-[(3,4-dichlorobenzyl)(methyl)amino]propyl}acetamide;
N-{3-[(3,4-Dichlorobenzyl)(methyl)amino]propyl}-2-(6-methoxybezoxazol-2-ylsulfanyl)acetamide;
N-{3-[(3,4-Dichlorobenzyl)(methyl)amino]propyl}-2-(1,6-dimethyl-1H-benzimidazol-2-ylsulfanyl)acetamide;
N-{3-[(3,4-Dichlorobenzyl)(methyl)amino]propyl}-2-(oxazolo[5,4-b]pyridin-2-ylsulfanyl)acetamide;
2-(6-Aminobenzthiazol-2-ylsulfanyl)-N-{3-[(3,4-dichlorobenzyl)(methyl)amino]propyl}acetamide;
2-(Benzthiazol-2-ylsulfanyl)-N-{3-[(3,4-dichlorobenzyl)(methyl)amino]propyl}acetamide;
N-{3-[(3,4-Dichlorobenzyl)(methyl)amino]propyl}-2-(6-methylbezoxazol-2-ylsulfanyl)acetamide;
N-{3-[(3,4-Dichlorobenzyl)(methyl)amino]propyl}-2-(thiazolo[5,4-b]pyridin-2-ylsulfanyl)acetamide;
2-(Benzoxazol-2-ylsulfanyl)-N-{3-[(3,4-dichlorobenzyl)(methyl)amino]propyl}acetamide;
N-{3-[(3,4 Dichlorobenzyl)(methyl)amino]propyl}-2-(5-methoxybenzothiazol-2-ylsulfanyl)acetamide;
N-{3-[(3,4-Dichlorobenzyl)(methyl)amino]propyl}-2-(6-ethoxybenzothiazol-2-ylsulfanyl)acetamide;
N-{3-[(3,4-Dichlorobenzyl)(methyl)amino]propyl}-2-(5-ethylaminothiazolo[5,4-d]pyrimidin-2-ylsulfanyl)acetamide;
N-{3-[(3,4-Dichlorobenzyl)(methyl)amino]propyl}-2-(5-ethylaminothiazolo[5,4-b]pyridin-2-ylsulfanyl)acetamide;
N-{3-[(3,4-Dichlorobenzyl)(methyl)amino]propyl}-2-(5-isopropylaminothiazolo[5,4-d]pyrimidin-2-ylsulfanyl)acetamide;
N-{3-[(3,4-Dichlorobenzyl)(methyl)amino]propyl}-2-(5-isopropylaminothiazolo[5,4-b]pyridin-2-ylsulfanyl)acetamide;
2-(5-Benzylaminothiazolo[5,4-b]pyridin-2-ylsulfanyl)-N-{3-[(3,4-dichlorobenzyl) (methyl)amino]propyl}acetamide;
2-(6-Aminobenzthiazol-2-ylsulfanyl)-N-{3-[(3,4-dichlorobenzyl)(methyl)amino]butyl}acetamide;
2-(6-Aminobenzthiazol-2-ylsulfanyl)-N-{3-[(3,4-dichlorobenzyl)(methyl)amino]propyl}butyramide;
N-{3-[(3,4-Dichlorobenzyl)(methyl)amino]propyl}-2-(6-methyl-1H-benzimidazol-2-ylsulfanyl)acetamide;
N-{3-[(3,4-Dichlorobenzyl)(methyl)amino]propyl}-2-(quinazolin-2-ylsulfanyl)acetamide;
2-(5-Benzylaminothiazolo[5,4-d]pyrimidin-2-ylsulfanyl)-N-{3-[(3,4-dichlorobenzyl) (methyl)amino]propyl}acetamide;
2-(6-Aminobenzthiazol-2-ylsulfanyl)-N-{2-[(3,4-Dichlorobenzyl)(methyl)amino]ethyl}acetamide;
3-(6-Aminobenzthiazol-2-ylsulfanyl)-N-{3-[(3,4-dichlorobenzyl)(methyl)amino]propyl}propionamide;
2-(6-Aminobenzthiazol-2-ylsulfanyl)-N-{3-[(3,4-dichlorobenzyl)(methyl)amino]propyl}acetamide;
2-(6-Aminobenzthiazol-2-ylsulfanyl)-N-{3-[(3,4-dichlorophenyl)propyl](methyl) amino-propyl}acetamide;
2-(6-Aminobenzthiazol-2-ylsulfanyl)-N-{3-[(3,4-dichlorobenzyl)(methyl)amino]butyl}acetamide;
N-{3-[(3,4-Dichlorobenzyl)(methyl)amino]-1-methylpropyl}-2-(6-methylbenzoxazol-2-ylsulfanyl)acetamide;
N-{3-[(3,4-Dichlorobenzyl)(methyl)amino]-1-methylpropyl}-2-(thiazolo[5,4-b]pyridin-2-ylsulfanyl)acetamide;
2-(6-Aminobenzthiazol-2-ylsulfanyl)-N-{3-[(3,4-dichlorobenzyl)(methyl)amino]-2-methylpropyl}acetamide;
2-(6-Aminobenzthiazol-2-ylsulfanyl)-N-{3-[(3,4-dichlorobenzyl)(methyl)amino]-1-methylpropyl}acetamide;
2-(6-Aminobenzthiazol-2-ylsulfanyl)-N-{3-[(3,4-dichlorobenzyl)(methyl)amino]propyl}-N-methylacetamide;
(+)N-{3-[(3,4-Dichlorobenzyl)(methyl)amino]-1-methylpropyl}-2-(6-methylbenzoxazol-2-ylsulfanyl)acetamide;
(−)N-{3-[(3,4-Dichlorobenzyl)(methyl)amino]-1-methylpropyl}-2-(6-methylbenzoxazol-2-ylsulfanyl)acetamide;
2-(6-Aminobenzthiazol-2-ylsulfanyl)-N-{3-[(3,4-dichlorobenzyl)(methyl)amino]propyl}-propionamide; and
N-{3-[(3,4-Dichlorobenzyl)(methyl)amino]propyl}-2-(4-methylbenzoxazol-2-yl)sulfinyl]acetamide; or
a salt, solvate or isomer thereof, salt of the solvate or isomer thereof, or solvate of the isomer thereof.

12. A method of treatment of a pathology in a patient wherein a CCR3 receptor plays a role in the development of the pathology comprising administering to the patient a pharmaceutically effective amount compound of the general formula (I) according to claim 1.

13. The method according to claim 12 wherein the pathology is selected from asthma, allergic rhinitis, atopic dermatitis, eczema, inflammatory bowel disease, ulcerative colitis, allergic conjunctivitis, multiple sclerosis, Crohn's disease, HIV-infection and diseases in conjunction with AIDS.

14. The compound of the general formula (III), 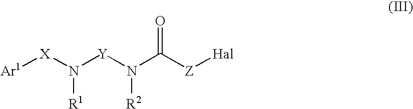
where Ar¹, X, Y, Z, R¹ and R² have the meanings as defined in claim 1 and Hal stands for halogen atom.
